SEQUENCE LISTING (31 sequences)

SEQ ID NO: 1 - protein, 585 aa, Homo sapiens
  UNSURE (1)..(585)
MAAVELEWIPETLYNTAISAVVDNYIRSRRDIRSLPENIQFDVYYKLYQQGRLCQLGSEFCELEVFAKVLRALDKRHLLHHCFQALMDHGVKVASVLAYSFSRRCSYIAESDAAVKEKAIQVGFVLGGFLSDAGWYSDAEKVFLSCLQLCTLHDEMLHWFRAVECCVRLLHVRNGNCKYHLGEETFKLAQTYMDKLSKHGQQANKAALYGELCALLFAKSHYDEAYKWCIEAMKEITAGLPVKVVVDVLRQASKACVVKREFKKAEQLIKHAVYLARDHFGSKHPKYSDTLLDYGFYLLNVDNICQSVAIYQAALDIRQSVFGGKNIHVATAHEDLAYSSYVHQYSSGKFDNALFHAERAIGIITHILPEDHLLLASSKRVKALILEEIAIDCHNKETEQRLLQEAHDLHLSSLQLAKKAFGEFNVQTAKHYGNLGRLYQSMRKFKEAEEMHIKAIQIKEQLLGQEDYEVALSVGHLASLYNYDMNQYENAEKLYLRSIAIGKKLFGEGYSGLEYDYRGLIKLYNSIGNYEKVFEYHNVLSNWNRLRDRQYSVTDALEDVSTSPQSTEEVVQSFLISQNVEGPSC

SEQ ID NO: 2 - DNA, 2285 bp, Homo sapiens
  mRNA (1)..(2285)
ttctgtcccactttttactcgggcctgcgtccgctgccgccgtccctcagtttgcccccggaggaggcagggcggccgtgccttctgccgtgcgcccgcgtggctgccaccgcccctccgaatcctccggggccgcagaggggttcgctacggagggaggtgggggccttcgggaggaggaggcggaggaggcggaggaggagggaaggaagatggcggccgtggaactagagtggatcccagagactctctataacaccgccatctccgctgtcgtggacaactacatccgctcccgccgagacatccgctccttgcccgagaacatccagtttgatgtttactacaagctttaccaacagggacgcttatgtcaactgggcagtgaattttgtgaattggaagtttttgctaaagtactgagagctttggataaaagacatttgcttcatcattgttttcaggctttgatggatcatggtgttaaagttgcttcagtcttggcctactcattcagtaggcggtgctcttatatagcagaatcagatgctgcagtaaaggaaaaagccattcaggttggctttgttttaggtggctttctttcagatgcaggctggtacagtgatgctgagaaagtttttctgtcctgccttcagttgtgtactctacacgatgagatgcttcattggtttcgtgcagtagaatgttgtgtgaggttgcttcatgtgcgaaatggaaactgcaaatatcatttgggtgaagaaacatttaaattagctcagacatatatggataaactatcaaaacatggccagcaagcaaataaagctgcactctatggagaactgtgtgcactcctatttgcaaaaagtcactatgatgaggcatacaaatggtgcatcgaggcaatgaaagaaattacagcaggcttaccagtgaaagttgtggtggatgtcttaagacaagcttctaaggcttgtgtagtaaaacgtgaatttaagaaggcagaacagttaattaaacatgcagtgtatttggcacgggatcattttggatccaaacacccaaaatattctgatacactgctagattatgggttctacttactcaatgtagataatatctgtcagtctgttgcaatttatcaggcagcccttgacattagacagtcagtgtttggtggcaaaaatatccacgtagcaacagctcatgaagatttggcctactcttcttatgtccaccagtatagctctgggaaatttgacaatgcactatttcatgcagaaagagctattggtatcattacccacatcctacctgaagatcatcttcttttggcttcttcaaagagggtgaaagcacttattttagaggagattgcaattgattgtcataataaggaaactgaacagaggctgcttcaagaagctcatgatttgcacctgtcttcactccaactagctaaaaaagcttttggggaatttaatgtacagactgcaaaacactatggaaaccttggaagactttatcagtcaatgagaaaatttaaggaagctgaagaaatgcacatcaaagcaattcagattaaagaacaacttcttggtcaagaagattatgaagtagccctttcagtgggacatctggcttctttatataattatgacatgaatcagtatgaaaatgctgagaaactttatttgcgatctatagcaattgggaagaaactttttggtgagggctacagtggactagaatatgattatcgaggtctcattaaactttacaactccattggaaattacgagaaagtgtttgaatatcacaatgttctgtctaactggaaccggttgcgagatcggcaatattcagtgacagatgctcttgaagatgtcagcaccagcccccagtccactgaagaagtggtgcagtccttcctgatttctcagaatgtcgagggaccgagctgctgagggaggacctcagttaaccaattaccttttcccggattccagggaattcatactgtgaaatcaaaaccatgttgttttggggggctggaatttgcattgaaacactggtccagtccattgaagaccctattttgggtgatccctatcttgcagaatgtctgtaggaataagcatatattcagttatattcagcatgtaccgcatgtgtaagtagtctggcccacattttcaacctagtagaacaaacaacaggaaatctttttttgttgtttttaaaaaattcattttgcagaaagcctgaaaaaaaaaaaaaaa

SEQ ID NO: 3 - protein, 920 aa, Homo sapiens
  UNSURE (1)..(920)
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 4 - DNA, 4314 bp, Homo sapiens
  Unsure (1)..(4314)
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaac

SEQ ID NO: 5 - protein, 433 aa, Homo sapiens
  UNSURE (1)..(433)
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 6 - DNA, 7134 bp, Homo sapiens
  Unsure (1)..(7134)
cgggcttgtgccgccgccgccgccgccgccgcccgggccaagtgacaaaggaaggaaggaagcgaggaggagccggccccgcagccgctgacagggctctgggctggggcaaagcgcggacacttcctgagcgggcaccgagcagagccgaggggcgggagggcggccgagctgttgccgcggacgggggagggggccccgagggacggaagcggttgccgggttcccatgtccccggcgaatggggaacagtcgaggagccgctgcctggggtctgaagggagctgcctccgccaccgccatggccgctggatccagccgccgcctgcagctgctcctggcgcaatgaggagaggagccgccgccaccgccaccgcccgcctctgactgactcgcgactccgccgccctctagttcgccgggcccctgccgtcagcccgccggatcccgcggcttgccggagctgcagcgtttcccgtcgcatctccgagccaccccctccctccctctccctccctcctacccatccccctttctcttcaagcgtgagactcgtgatccttccgccgcttcccttcttcattgactcggaaaaaaaatccccgaggaaaatataatattcgaagtactcattttcaatcaagtatttgcccccgtttcacgtgatacatatttttttaggatttgccctctcttttctctcctcccaggaaagggaggggaaagaattgtattttttcccaagtcctaaatcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaagagaaaaaaatcctgttcattttagtgttcaatttttttattattattgttgttcttatttaaccttgtaaaatatctataaatacaaaccaatttcattgtattctcactttgagggagatccagggggtgggaggggttgtggggagggggaaagcggagcactagaacatacaatctctctcccacgacaatctttttttattaaaagtctgctgttgtatactttaaaaacaggactcctgcctcatgccccttccacaaaagaagaaaacctttttctgtgctgatgggtttttttgaactttgttttcttttaaagtctagtgtgagactttggtatagtgcacagcttgaaattggttgggagcttagcaggtataactcaacggggacttaaatgtcacttgtaaaattaatccatatcttcgggtatttatagacttgcctttggcatgttggtggcaggtgtggcagacaaagaaatgtgtatcattcgtaacccagggaggtcaataaagtttggaactctacagggaagattcttagtagatttgttaaggttttgttttgctctcagttagtgctagtgatgtagaggcttgtacaggaggctgccagaggggaagcagcaagcaagactcaggcacacatgctctacaggtggctctttgtttgcctgaccaaagttctttgcaaatcttagcacagtttcaaactagtgacctgggaggagatggaaggggtgttgagcaggctgagctagctgctgaggtcaaaggctgatgagcccagaggaaggggacaggtcagggatacatctcaccactgtgaataagtttgtccagatttttttctaaagttacttcccttggaaagatacacttgagaggacattgtagttaaataatgtgaactgtaacagtcatctactggtttatttttcatattttttaattgaaaattgagcttgcagaaatagccacattctacacatagttctaattttaaatccaaatctagaatctgtatttaatttgttttttaacctcatgctttttacatttatttattgatgcatgtcagatggtagaaatattaaaaactacacatcagaatgatacagtcacttatacctgctgactttataggaaagctgatgatataaatgtgtgtatatatgttatatatacatatattcaatactgcctttttttttgtctacagtatcaaaattgactggttgaagcatgagaagaatgtttcccccacacccagttaagagtttttgtgtctgttttctttgtgtatcagtgaacgatgttaagaatcagtctctctttttgaagaaaaagcaatattccttggaaagcaaggagaattgaaggactatgtttgccgtgaggaaatagattttcatgactagtttgttttatacttttaaggttggcatctatgtgggccttatatactctaaaatgaactttagtcaccttggtgcttatgggccattacttgacctatgaatctttaaggcacaatcagttgtactttacatttaaagatcacttgagtgatggccgcctttccctcctacccgctccttccccacatgccttccaaggttagctggtaactgtagggctgcagagctgagcccatggttgtgtgtaacttgccctcaccctcctcattgccaccttaggtcactttatgggtctcgtcctccagagggttcggaagtggagtctgttggcagccctcctgcaggccctagcaccctgtcctgctccttaactgtgtgtgtgactctccaagagagttgtcctgcctgctgaagtgaaccagtacccagaaagacaactgtgagccatcttggttttcactcgctgtttagctgaggtcttgggccacaaaaggggtttcacaaacctctggatatatcagagtttatgagaaaggaaacatgctcagtcaaaccaaatcaaacaaattgaattttatgttttataaagtgcttctgaaagctaagatttgaaagaagtctgaaatcaaagtatttggcagcataactccttaaaggtagtggcgttgatagaccattttcagacagaatttataaagaatctgaaaaggcaggtctgtgatagagaaatggacctgcattcagatccaactgcccagcaagcgtttggatgcagacactgctctggacgtggtatactccccagagtccataaaaatcagtgcttattttaggaaacaggttgccccccacaactggggtaaaagaagagagaaaagtcacgcttttctctcatttcattgtgtgtgcatgtgtgcgtgtgtgtgtgtgtgtgtgtgtgctgagatgtgtgatttttctttctcaaggatcatggtgggatcacagaactcttttatacaagtgagatccaggtctctgaatatctttttgtatataataataataaaaagctcctcaccaaattcaagcttgtacattatattttctttctgtgtttttaaatttaagttttattgttttgtatgtaaatatgtggacccaggaactgttattaatgagcaaaaagttactgttcagggcagtgattctgtttaataatcagacaaaatgtagacgagctttttaaagccatatagttttaactctgtacagtaggtaccggcctgtattattgtaacaataactctagcaatgtatagtgtatctatatagtttggagtgccttcgcttccatgtgtttttttttttaatttgttcttttttaaattttaattggtttcctttatccatgtctccctgtccaccccctttccctttgaaataataactcactcataacagtatctttgccccttccacagttaagtttcagtgataccatactcaggagtgggaagaggaaatcatattcgtaatttcatttcgttgaagccctgcctttgttttggttctgaatgtctttcctcctcggtagcagtgagaccggtttcatttcatacttagtccattcagggacttagtgtagcaccagggagccctagagctggaggatatcgaatagattaaattttgctcgtctcttccacaagccctaaccatgggtcttaaaaacagcagattctgggagccttccatgctctctctctctcctcttttatctacttccctcccaaatgagagagtgacagagaattgtttttttataaatcgaagtttcttaatagtatcaggttttgatacgtcagtggtctaaaatgctatagtgcaattactagcagttactgcacggagtgccaccgtgccaatagaggactgttgttttaacaagggaactcttagcccatttcctccctcccgccatctctacccttgctcaatgaaatatcattttaatttcttttaaaaaaaatcagtttaattcttactgtgtgcccaacacgaaggccttttttgaaagaaaaatagaatgttttgcctcaaagtagtccatataaaatgtcttgaatagaagaaaaaactaccaaaccaaaggttactatttttgaaacatcgtgtgttcattccagcaaggcagaagactgcaccttctttccagtgacatgctgtgtcattttttttaagtcctcttaatttttagacacatttttggtttatgttttaacaatgtatgcctaaccagtcatcttgtctgcaccaatgcaaaggtttctgagaggagtattctctatccctgtggatatgaagacactggcatttcatctatttttccctttcctttttaaaggatttaactttggaatcttccaaaggaagtttggccaatgccagatccccaggaatttggggggttttctttcttttcaactgaaattgtatctgattcctactgttcatgttagtgatcatctaatcacagagccaaacacttttctcccctgtgtggaaaagtaggtatgctttacaataaaatctgtcttttctggtagaaacctgagccactgaaaataaaagagacaactagaagcacagtagagtcccagactgagatctacctttgagaggctttgaaagtaatccctggggtttggattattttcacaagggttatgccgttttattcaagtttgttgctccgttttgcacctctgcaataaaagcaaaatgacaaccagtacataaggggttagcttgacaaagtagacttccttgtgttaatttttaagtttttttttccttaactatatctgtctacaggcagatacagatagttgtatgaaaatctgcttgcctgtaaaatttgcatttataaatgtgttgccgatggatcacttgggcctgtacacataccaattagcgtgaccacttccatcttaaaaacaaacctaaaaaacaaaatttattatatatatatatatatatatataaaggactgtgggttgtatacaaactattgcaaacacttgtgcaaatctgtcttgatataaaggaaaagcaaaatctgtataacattattactacttgaatgcctctgtgactgatttttttttcattttaaatataaacttttttgtgaaaagtatgctcaatgttttttttccctttccccattcccttgtaaatacattttgttctatgtgacttggtttggaaatagttaactggtactgtaatttgcattaaataaaaagtaggttagcctggaaatgaaattaaaaaaaaaaaaaaaaaa

SEQ ID NO: 7 - protein, 391 aa, Homo sapiens
  unsure (1)..(391)
MKCLVTGGNVKVLGKAVHSLSRIGDELYLEPLEDGLSLRTVNSSRSAYACFLFAPLFFQQYQAATPGQDLLRCKILMKSFLSVFRSLAMLEKTVEKCCISLNGRSSRLVVQLHCKFGVRKTHNLSFQDCESLQAVFDPASCPHMLRAPARVLGEAVLPFSPALAEVTLGIGRGRRVILRSYHEEEADSTAKAMVTEMCLGEEDFQQLQAQEGVAITFCLKEFRGLLSFAESANLNLSIHFDAPGRPAIFTIKDSLLDGHFVLATLSDTDSHSQDLGSPERHQPVPQLQAHSTPHPDDFANDDIDSYMIAMETTIGNEGSRVLPSISLSPGPQPPKSPGPHSEEEDEAEPSTVPGTPPPKKFRSLFFGSILAPVRSPQGPSPVLAEDSEGEG

SEQ ID NO: 8 - DNA, 2102 bp, Homo sapiens
  unsure (1)..(2102)
gcgcgggaagggaccccggacccggaggtcgcggagagctgggcagtgttggccgctggcggagcgctggggcagcatgaagtgcctggtcacgggcggcaacgtgaaggtgctcggcaaggccgtccactccctgtcccgcatcggggacgagctctacctggaacccttggaggacgggctctccctccggacggtgaactcctcccgctctgcctatgcctgctttctctttgccccgctcttcttccagcaataccaggcagccacccctggtcaggacctgctgcgctgtaagatcctgatgaagtctttcctgtctgtcttccgctcactggcgatgctggagaagacggtggaaaaatgctgcatctccctgaatggccggagcagccgcctggtggtccagctgcattgcaagttcggggtgcggaagactcacaacctgtccttccaggactgtgagtccctgcaggccgtcttcgacccagcctcgtgcccccacatgctccgcgccccagcacgggttctgggggaggctgttctgcccttctctcctgcactggctgaagtgacgctgggcattggccgtggccgcagggtcatcctgcgcagctaccacgaggaggaggcagacagcactgccaaagccatggtgactgagatgtgccttggagaggaggatttccagcagctgcaggcccaggaaggggtggccatcactttctgcctcaaggaattccgggggctcctgagctttgcagagtcagcaaacttgaatcttagcattcattttgatgctccaggcaggcccgccatcttcaccatcaaggactctttgctggacggccactttgtcttggccacactctcagacaccgactcgcactcccaggacctgggctccccagagcgtcaccagccagtgcctcagctccaggctcacagcacaccccacccggacgactttgccaatgacgacattgactcttacatgatcgccatggaaaccactataggcaatgagggctcgcgggtgctgccctccatttccctttcacctggcccccagccccccaagagccccggtccccactccgaggaggaagatgaggctgagcccagtacagtgcctgggactcccccacccaagaagttccgctcactgttcttcggctccatcctggcccctgtacgctccccccagggccccagccctgtgctggcggaagacagtgagggtgaaggctgaaccaagaacctgaagcctgtacccagaggccttggactagacgaagccccagccagtggcagaactgggtctctcagccctggggatcagaaaggtgggcttgctggagctgagctgtttcactgcctctcgcaggccccagctggctgtcactgtaaagctgtcccacagcggtcgggcctgggccgttatctccccacaacccccagccaatcaggactttccagacttggccctgaactactgacgttcctacctcttatttctcattgagcctcaggctatactccagctggccaaggctggaaacctgtctccctcaggctcaccttcctaaggaaaatgtcatagtaggtgctgctggcccctggtgatccagcttctctgccaatcatgacctgttccttcctgaagtcctgggcatgcatctgggacccccgtggagctgacaagttttccttgctttcctgatactctttggcgctgacttggaattctaagagccttggacccgagtgtgtggctagggttgccctggctggggcccggtgccgagactcccaagcggctctgtgcagaagagctgccaggcagtgtcttagatgtgagacggaggccatggcgagaatccagctttgacctttattcaagagaccagatgggttgccccaggatccggctgccagccctgaggccaagcacggctggagacccacgacctggcctgccgttgccctgagctgcagcctcggccccaggatcctgctcacagtcaccgcaggtgcaggcaggaagcagccctgggggactggacgctgctattgattcattaaaaaaagaaaagaaaaat

SEQ ID NO: 9 - DNA, 22 bp, Artificial Sequence
  fragment of Androgen Receptor siRNA
gggcccctggatggatagctac

SEQ ID NO: 10 - DNA, 21 bp, Artificial Sequence
  fragment of androgen receptor siRNA
gtagctatccatccaggggcc

SEQ ID NO: 11 - DNA, 49 bp, Unknown
  androgen receptor siRNA
gggcccctggatggatagctacctcgaggtagctatccatccaggggcc

SEQ ID NO: 12 - DNA, 54 bp, Artificial Sequence
  androgen receptor siRNA with polyT after U6 promoter
tttttgggcccctggatggatagctacctcgaggtagctatccatccaggggcc

SEQ ID NO: 13 - protein, 483 aa, Homo sapiens
  unsure (1)..(483)
MATIEEIAHQIIEQQMGEIVTEQQTGQKIQIVTALDHNTQGKQFILTNHDGSTPSKVILARQDSTPGKVFLTTPDAAGVNQLFFTTPDLSAQHLQLLTDNSPDQGPNKVFDLCVVCGDKASGRHYGAVTCEGCKGFFKRSIRKNLVYSCRGSKDCIINKHHRNRCQYCRLQRCIAFGMKQDSVQCERKPIEVSREKSSNCAASTEKIYIRKDLRSPLTATPTFVTDSESTRSTGLLDSGMFMNIHPSGVKTESAVLMTSDKAESCQGDLSTLANVVTSLANLGKTKDLSQNSNEMSMIESLSNDDTSLCEFQEMQTNGDVSRAFDTLAKALNPGESTACQSSVAGMEGSVHLITGDSSINYTEKEGPLLSDSHVAFRLTMPSPMPEYLNVHYIGESASRLLFLSMHWALSIPSFQALGQENSISLVKAYWNELFTLGLAQCWQVMNVATILATFVNCLHNSLQQDAKVIAALIHFTRRAITDL

SEQ ID NO: 14 - protein, 596 aa, Homo sapiens
  unsure (1)..(596)
MTSPSPRIQIISTDSAVASPQRIQIVTDQQTGQKIQIVTAVDASGSPKQQFILTSPDGAGTGKVILASPETSSAKQLIFTTSDNLVPGRIQIVTDSASVERLLGKTDVQRPQVVEYCVVCGDKASGRHYGAVSCEGCKGFFKRSVRKNLTYSCRSNQDCIINKHHRNRCQFCRLKKCLEMGMKMESVQSERKPFDVQREKPSNCAASTEKIYIRKDLRSPLIATPTFVADKDGARQTGLLDPGMLVNIQQPLIREDGTVLLATDSKAETSQGALGTLANVVTSLANLSESLNNGDTSEIQPEDQSASEITRAFDTLAKALNTTDSSSSPSLADGIDTSGGGSIHVISRDQSTPIIEVEGPLLSDTHVTFKLTMPSPMPEYLNVHYICESASRLLFLSMHWARSIPAFQALGQDCNTSLVRACWNELFTLGLAQCAQVMSLSTILAAIVNHLQNSIQEDKLSGDRIKQVMEHIWKLQEFCNSMAKLDIDGYEYAYLKAIVLFSPDHPGLTSTSQIEKFQEKAQMELQDYVQKTYSEDTYRLARILVRLPALRLMSSNITEELFFTGLIGNVSIDSIIPYILKMETAEYNGQITGASL

SEQ ID NO: 15 - DNA, 2029 bp, Homo sapiens
  unsure (1)..(2029)
gaattcgggcccgtcggctttcttcaaccctctcttcccggagcgcccccaatccacgagtggcagccgcgggactgtcgcgtcggcgcccgacgcggagtcagcaggggcgaaaagcggtagatcatggcaaccatagaagaaattgcacatcaaattattgaacaacagatgggagagattgttacagagcagcaaactgggcagaaaatccagattgtgacagcacttgatcataatacccaaggcaagcagttcattctgacaaatcacgacggctctactccaagcaaagtcattctggccaggcaagattccactccgggaaaagttttccttacaactccagatgcagcaggtgtcaaccagttattttttaccactcctgatctgtctgcacaacacctgcagctcctaacagataattctccagaccaaggaccaaataaggtttttgatctttgcgtagtatgtggagacaaagcatcaggacgtcattatggagcagtaacttgtgaaggctgcaaaggattttttaaaagaagcatccgaaaaaatttagtatattcatgtcgaggatcaaaggattgtattattaataagcaccaccgaaaccgctgtcaatactgcaggttacagagatgtattgcgtttggaatgaagcaagactctgtccaatgtgaaagaaaacccattgaagtatcacgagaaaaatcttccaactgtgccgcttcaacagaaaaaatctatatccgaaaggaccttcgtagcccattaactgcaactccaacttttgtaacagatagtgaaagtacaaggtcaacaggactgttagattcaggaatgttcatgaatattcatccatctggagtaaaaactgagtcagctgtgctgatgacatcagataaggctgaatcatgtcagggagatttaagtacattggccaatgtggttacatcattagcgaatcttggaaaaactaaagatctttctcaaaatagtaatgaaatgtctatgattgaaagcttaagcaatgatgatacctctttgtgtgaatttcaagaaatgcagaccaacggtgatgtttcaagggcatttgacactcttgcaaaagcattgaatcctggagagagcacagcctgccagagctcagtagcgggcatggaaggaagtgtacacctaatcactggagattcaagcataaattacaccgaaaaagaggggccacttctcagcgattcacatgtagctttcaggctcaccatgccttctcctatgcctgagtacctgaatgtgcactacattggggagtctgcctccagactgctgttcttatcaatgcactgggcactttcgattccttctttccaggctctagggcaagaaaacagcatatcactggtgaaagcttactggaatgaactttttactcttggtcttgcccagtgctggcaagtgatgaatgtagcaactatattagcaacatttgtcaattgtcttcacaatagtcttcaacaagatgccaaggtaattgcagccctcattcatttcacaagacgagcaatcactgatttataaatgcttaactatagaatggcttatgactacccaaaacagtgccccatcaacaaatggggaaaattgccttttgagctcaggaataatttataaattggggactaccttttagttctttagcatattctatttcttattgttttatataatttttaaatcatttgcttcctccttatgtttaacagcagaggggtaatcaccttaaaatgtcatcaaaaatagatctactagaaggcagcatcacattcccatcttacttatggactcctacccctggttcatgtcttatatgcctgtaatggttataaagcctaccttcaggaaagctatggttgactaattactaatggatgggttttaaacatgtccctctacaataaattaaaatctttcaatgtttgaatataatgtggaggtgtttacctgagggcctctctatctccccgaattc

SEQ ID NO: 16 - DNA, 6450 bp, Artificial Sequence
  synthetic construct
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 17 - DNA, 23 bp, Unknown
  hRad9, forward primer
cgctgtaagatcctgatgaagtc

SEQ ID NO: 18 - DNA, 19 bp, Unknown
  hRad9, reverse primer
tgcctcctcctcgtggtag

SEQ ID NO: 19 - DNA, 21 bp, Unknown
  18s rRNA, forward primer
tgccttccttggatgtggtag

SEQ ID NO: 20 - DNA, 21 bp, Unknown
  18s rRNA, reverse primer
cgtctgccctatcaactttcg

SEQ ID NO: 21 - protein, 920 aa, Unknown
  mutant androgen receptor R614H
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFHRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 22 - protein, 12 aa, unknown
  small hRad9 peptide
PKKFRSLFFGSI

SEQ ID NO: 23 - protein, 19 aa, Unknown
  small FXXLL peptide
HPTHSSRLWELLMEATPTM

SEQ ID NO: 24 - DNA, 20 bp, unknown
  Gadd45a, primer 1
tgagctgctgctactggaga

SEQ ID NO: 25 - DNA, 20 bp, Unknown
  Gadd45a, primer 2
tgtgatgaatgtgggttcgt

SEQ ID NO: 26 - DNA, 20 bp, Unknown
  Gadd45b, primer 1
attgacatcgtccgggtatc

SEQ ID NO: 27 - DNA, 20 bp, Unknown
  Gadd45b, primer 2
tgacagttcgtgaccaggag

SEQ ID NO: 28 - DNA, 18 bp, Unknown
  MnSOD, primer 1
ctccaggcagaagcacag

SEQ ID NO: 29 - DNA, 21 bp, Unknown
  MnSOD, primer 2
gatatgaccaccaccattgaa

SEQ ID NO: 30 - DNA, 20 bp, Unknown
  Thioredoxin2, primer 1
cagcctctggcacatttcct

SEQ ID NO: 31 - DNA, 21 bp, Unknown
  Thioredoxin2, primer 2
gttcggcttctggtttccttt

(19) United States
(12) Patent Application Publication
(10) Pub. No.: US 2010/0048676 A1
(43) Pub. Date: Feb. 25, 2010

(54) Non-androgen dependent roles for androgen receptor in liver cancer

(75) Inventor: Chawnshang Chang, Pittsford, NY (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: University of Rochester, Rochester, NY (US)

(21) Appl. No.: 12/535,487

(22) Filed: Aug. 4, 2009

Related U.S. Application Data
(60) Provisional application No. 61/086,151, filed on Aug. 4, 2008.
(60) Provisional application No. 61/086,256, filed on Aug. 5, 2008.

(51) Int. Cl. A61K 31/105; G01N 33/53; A61K 31/7088; A61P 35/00

(52) U.S. Cl. 514/44 A; 435/7.21

(57) ABSTRACT

Disclosed are compositions and methods for modulating AR activity, such as non-androgen dependent AR activity. Also disclosed are compositions and methods for diagnosing beast cancer and for inhibiting liver cancer growth. In addition, disclosed are methods for identifying molecules that inhibit AR in non-androgen dependent ways.

This application claims priority to U.S. Application No. 61/086,151, filed Aug. 4, 2008 and U.S. Application No. 61/086,256, filed Aug. 5, 2008. U.S. Application No. 61/086,151, filed Aug. 4, 2008 and U.S. Application No. 61/086,256, filed Aug. 5, 2008 are hereby incorporated herein by reference in their entirety.

### I. ACKNOWLEDGEMENTS

This invention was made with government support under federal grant CA122295 awarded by the National Institutes of Health and the George H. Whipple Professorship Endowment. The Government has certain rights to this invention.

Please incorporate-by-reference the material in the text file named 24376.41.8403 Sequence Listing ST25, created on Sep. 18, 2009, as a 73 kilobyte file per 37 CFR 1.52(e)(5).

### II. BACKGROUND OF THE INVENTION

Androgen receptor (AR) is a member of the steroid hormone superfamily of nuclear receptors. Androgen receptor has been implicated in many cancers in an androgen dependent way. Disclosed herein androgen receptor is also involved in the development of liver tissue and in the progression of liver cancers in androgen independent ways. Disclosed are ways of treating liver cancer and metastatic liver cancer that do not involve or are in addition to androgen ablation therapy.

### III. SUMMARY OF THE INVENTION

In accordance with the purposes of this invention, as embodied and broadly described herein, this invention, in one aspect, relates to compositions and methods related to androgen receptor and methods of inhibiting cancer.

It is to be understood that both the foregoing general description and the following detailed description are exemplary and explanatory only and are not restrictive of the invention, as claimed.

### IV. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1. AR expression in human livers and generation of mice lacking AR in hepatocyte only and serum testosterone level characterization. (A) Hematoxylene & Eosin staining (upper panel) and the nuclear AR staining (lower panel) of a dysplastic liver. (B) AR nuclear staining in tumor lesion (T), while less in non-tumor (non-T) (upper panel); AR nuclear staining in tumor margin (lower panel). (C, D) Immunohistochemical staining of AR in 28-weeks-old DEN-induced AR^{+/y} and L-AR^{-/y} liver tumor. AR positive staining is brown in AR^{+/y} transformed foci with higher magnification of the indicated area shown in the inset (C). In contrast, there is no positive signal in transformed foci of L-AR^{-/y} liver with higher magnification image of the indicated area is shown in the inset (D). (E) Serum Testosterone level measured by ELISA assay. * represents a significant difference (p<0.05) between male and female; # indicates a significant difference (p<0.05) between T-AR^{-/y} and AR^{+/y}.

FIG. 2. AR effect on hepatocarcinogenesis. (A) HCC incidence of mice. 20 mg/kg/mice of DEN was injected I.P. into 12-days mouse pups. After various time periods, 20-, 24-, 28-, 32-, 36-, and 40-weeks, mice were sacrificed and hepatocarcinogenesis was observed in all mice. A tumor was defined as positive if it could be observed by the naked eye. WT mice (AR^{+/y} and AR^{+/+}) are represented as solid line [symbol] T-ARKO (T-AR^{-/y} and T-AR^{-/-}) as rectangle dashed line [symbol] L-ARKO (L-AR^{-/y} and L-AR^{-/-}) as circle dashed line [symbol] (B) Tumor foci numbers in 36-weeks DEN-induced male mice decreased in T-AR^{-/y} and L-AR^{-/y} compared to AR^{+/y} (p<0.05). (C) Liver weight//Body weight (LW/BW) ratio in 36-weeks DEN-induced male mice decreased in T-AR^{-/y} and L-AR^{-/y} compared to AR^{+/y} (p<0.05). (D) BrdU (proliferation) and TUNEL (apoptosis) staining in 36-weeks DEN-induced male mice livers. BrdU positive proliferation stains were found to decrease while TUNEL stains increased in T-AR^{-/y} and L-AR^{-/y} compared to AR^{+/y} mice liver. These experiments were from 3 mice and 3 different sections of livers from each genotype. The numbers of positive stains from each slide were pooled from photographed image of sections (3 area/slide; under 10×10 magnification). (E) Cell growth analysis using MTT assay on the cells derived from AR^{+/y} primary liver tumor culture in 55-weeks DEN-induced AR^{+/y} mice. Cells within 3 passages of subculture were used, treated with ethanol (EtOH) or DHT at different concentrations (1 and 10 nM). Cell growth was monitored for a maximum of 8 days and harvested for MTT assay. Values from background readings at 650 nm were subtracted and pooled all MTT assay results from 5 independent experiments.

FIG. 3. AR promotes anchorage-dependent and -independent cell growth. (A) Apoptotic cells in SKpar and SKAR3 cells. Cells were plated and treated with EtOH or 10 nM DHT for 48 hrs then stained with PI for cell apoptosis using flowcytometry. * indicated significant difference between SKpar and SKAR3 cells (p<0.05). (B) Androgen and AR effect on anchorage-dependent cell growth. SKpar and SKAR3 cells were treated with EtOH or 10 nM DHT for 4 days and counted the cell numbers to measure cell growth. * indicates the significant difference between SKpar and SKAR3EtOH treatments. ** indicates the significant difference between SKAR3-DHT to SKAR3-EtOH and SKAR3-DHT to SKpar-DHT (p<0.05). (C) Anchorage-independent cell growth of SKpar and SKAR3 cells. Cell clusters greater than 50 cells were counted as a positive clone. All data were from 3-5 independently repeated experiments that showed similar results with the error bar indicating±SD of pooled results.

FIG. 4. AR promotes cellular oxidative stress through down-regulating ROS enzymes. (A) Oxidative attacked cellular protein decreased in L-AR^{-/y} liver tumors compared to AR^{+/y} liver. Protein from 36-week DEN-induced mice livers were derivatized to form carbonylated groups that can be recognized by a specific antibody. The derivatized samples were dot blotted on PVDF membrane and stained for carbonyl group and actin antibody. Representative results from AR^{+/y} mice and L-AR^{-/y} membrane blots are shown in the left panels, and the quantitative results from three independent blotted membranes of different mice in the right panel that show a similar pattern. * Indicates significant difference (p<0.05). (B) SKpar and SKAR3 cells were treated with 200 μM H₂O₂ or 1 nM DHT for 24 hrs and measured ROS level. Three independent experiments were performed and pooled (C) IHC staining of DNA damage marker, 8-oxoG, in 36-weeks DEN-induced mice liver tumors. Positive staining (green spot) of 8-oxoG is more abundant in AR^{+/y} (left panel) than L-AR^{-/y} (middle panel) liver tumor. Three liver tumors with 3 different sectioned slides were examined and signals were analyzed, and quantitated using NIH-image software. Quantitated result are shown in right panel. Significant difference in AR^{+/y} and L-AR^{-/y} is indicated using * (p<0.05).

FIG. 5. AR suppresses p53 and down-stream target genes. (A) p53 protein expression in 36-week DEN-induced mouse HCC livers. p53 expression was measured with immunoblotting and it was shown in the quantitative results that p53 expression in the AR^{+/y} mice was lower than L-AR^{-/y}. GAPDH served as loading control. (B) AR, p53 and p21 protein expression in 36-weeks DEN-injected mouse normal livers. p53 expression was measured with immunoblotting that AR^{+/y} mice is lower than L-AR^{-/y}. β-Actin served as loading control. Higher expression of p53 and p21 proteins were detected in L-AR^{-/y} livers compared to AR^{+/y} (n=4 of each group). (C) Gadd45α and β protein expression were examined using specific antibodies, and compared in AR^{+/y} and L-AR^{-/y} liver tumors. Quantitation of Gadd45α and β protein expression in right panel. * indicates significant difference in AR^{+/y} and L-AR^{-/y} (p<0.05).

FIG. 6. Targeting AR as therapeutic strategy. (A) Establishment of AR siRNA stable transfectants of human SKAR3 cells. Scrambled siRNA (SKAR3-sc, lane 3) and different siRNA targeting AR (SKAR3-si1; SKAR3-si2; SKAR3-si3) stable transfectants derived from SKAR3 cells. LNCaP and SK-Hep1 cells served as positive and negative controls of AR expression, respectively. (B) AR transactivation activity was used to examine the knockdown efficiency of AR siRNA in the SKAR3 cells. The SKAR3-si1 cells were used to compare with SKAR3-sc cells and treated with EtOH and 1 nM DHT for 24 hrs after ARE(4)-luciferase transfection. The readings were normalized with the read out of pRL-TK cotransfection and pooled three individual experiments. (C) AR siRNA effect on SKAR3 cell growth. SKAR3-sc and SKAR3-si1 cells were treated with EtOH or 1 nM DHT, then observed cell growth by counting cells on different days. (D) ASC-J9 effect on SKAR3 and SKAR7 cells. Cells were plated and cultured with EtOH, 10 nM DHT and 5 μM ASC-J9 for different days and examined cell growth using MTT assay. (E) ASC-J9 effect on SKAR3 cell apoptosis and proliferation. Cells were cultured with 10 nM DHT, or 5 μM ASC-J9 for 24 hrs, then detached, stained with PI, assayed immediately by flowcytometry to observe cell apoptosis. (F) Primary cells were derived from 55-weeks DEN-induced AR^{+/y} liver tumors and cultured ex vivo. Cells were treated with ASC-J9 or cotreated with 10 nM DHT for 8 days, then harvested for MTT assay. The result represents three independent experiments. (G) ASC-J9 suppressed liver cancer growth in vivo. Primary cells were derived from 55-week DEN-induced AR^{+/y} liver tumors and subcutaneously inoculated into nude mice (2×10⁶ cells/site) flank. After 3 weeks, we IP injected mice with ASC-J9 (50 mg/kg/mice) twice per-week for 17 wks. Six injection sites from 3 mice were measured and pooled. Solvent (DMSO) group is shown as solid line, and ASC-J9 group is shown as dashed line.

FIG. 7. Generation of mice lacking AR in whole body or hepatocyte only and serum testosterone level characterization. (A) Mating strategy to generate mice lacking AR in the whole body (T-AR^{-/y}; T-AR^{-/-}) or hepatocyte only (L-AR^{-/y}; L-AR^{-/-}). (B) DNA product amplified by specific loxP-AR and Alb-Cre primers from mice tail snips to confirm genotype. The 550-bp products are loxP containing (flox) allele and 480-bp is WT allele, and 100-bp is Alb-Cre. (C) PCR product amplified by AR exon2-3 specific primers to differentiate truncated AR from different organs of L-AR^{-/y} mice. Te: testis; Li: liver; Sp: spleen; Br: brain; Ad: Adipose; Ki: kidney.

FIG. 8. Liver weight of non-DEN injected mice. A, B. The 16-wks old male mice liver from wild-type (AR^{+/y}), and L-ARKO (L-AR^{-/y}) were measured (A). Female liver of wild-type (AR^{+/+}), and L-ARKO (L-AR^{-/-}) from 16-wks mice were measured as well. The results show no significant differences between genotypes indicating AR doesn't influence static liver growth.

FIG. 9. Establishment of human HCC AR stable clones. (A) Establishment of AR stably-transfected cell lines from human SK-Hep1 HCC cells. AR protein abundance of different homogeneous colonies of AR stable transfectants (SKAR1; SKAR3; and SKAR7) were all much higher compared to SKpar (vector transfectant). LNCaP and DU145 prostate cancer cell lines served as positive and negative controls of AR expression respectively. (B) We treated SKpar and SKAR3 cells with EtOH or 1 nM DHT and examined cell lysates with ARE-driven luciferase assay. (C) Alpha-fetoprotein (AFP) protein expression can be up-regulated by androgen and AR signals.

FIG. 10. Examination of ROS reducing gene mRNA expression using SKpar and SKAR cells, (A, B) We plated cells and treated as indicated for 24 hrs. We examined Thioreducin-2 (A) and SOD2 (B) mRNA using Q-PCR as described in Methods. The data are means±SD from three independent experiments. * indicates significant difference comparing H₂O₂ and DHT-H₂O₂ treatments in SKAR3 cells (p<0.05).

FIG. 11. AR suppresses p53 stability and inhibits Gadd45α/β transcription using SKAR3 cells. (A) p53 protein expression of SKpar and SKAR3 cells. We treated SKpar and SKAR3 cells with 10 nM DHT for 2, 4, and 8 hrs and measured protein abundance by immunoblotting assay using p53 specific antibody. GAPDH served as loading control. (B) Quantitative result showed the down-regulation of p53 by androgen and AR signal in a time-dependent manner. (C) We measured Gadd45α promoter activity (C) in SKpar and SKAR3 cells under 24 hrs EtOH and 10 nM DHT treatments. (D) We also measured Gadd45β mRNA by Q-PCR using Gadd45β specific primers. We treated SKAR3 cells with either 200 mM H₂O₂ or 1 nM DHT for 24 hrs, then harvested and measured RNA. The data were from 3 independent experiments.

FIG. 12. AR suppress H₂O₂-induced apoptotic intrinsic pathway through regulation of p53. (A, B) p53 (A) and Bcl-2 (B) protein were measured under H₂O₂ treatment. Cells were either treated with vehicle or 200 μM H₂O₂ for different time periods. (C) We tested H₂O₂ effect on cell survival in SKpar and SKAR3 cells using MTT assay. Cells were treated with 200 μM H₂O₂ for 24 hrs and then incubated with MTT (5 μM) for 1 hr. After incubation, cells were harvested for analysis. The readings from H₂O₂ treated cells were normalized by the vehicle treated cells.

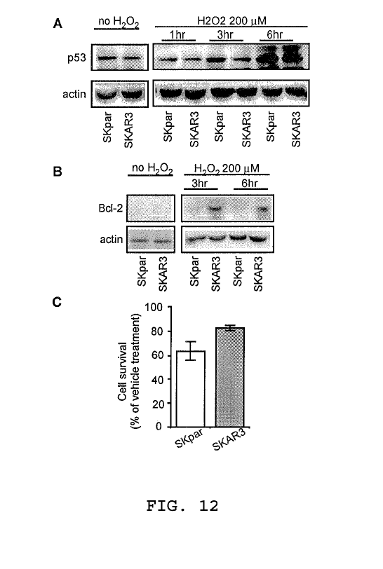

### V. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art, which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

“Coapplication” is defined as the application of one or more substances simultaneously, such as in the same formulation or consecutively, within a time frame such that each substance is active during a point when the other substance or substances are active.

The terms “higher,” “increases,” “elevates,” or “elevation” or variants of these terms, refer to increases above basal levels, e.g., as compared to a control. The terms “low,” “lower,” “reduces,” or “reduction” or variation of these terms, refer to decreases below basal levels, e.g., as compared to a control. For example, basal levels are normal in vivo levels prior to, or in the absence of, or addition of an agent such as an agonist or antagonist to activity.

The terms “control” or “control levels” or “control cells” are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. The subject can also be a non-human.

“Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Throughout the description and claims of this specification, the word “comprise” and variations of the word, such as “comprising” and “comprises,” means “including but not limited to,” and is not intended to exclude, for example, other additives, components, integers or steps.

The term “cell” as used herein also refers to individual cells, cell lines, or cultures derived from such cells. A “culture” refers to a composition comprising isolated cells of the same or a different type. The term co-culture is used to designate when more than one type of cell are cultured together in the same dish with either full or partial contact with each other.

When used with respect to pharmaceutical compositions, the term “stable” is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS AND METHODS

The liver is the largest gland in the body, and is typically situated slightly below the diaphragm and anterior to the stomach. It has two lobes which are wedge-shaped. Two blood vessels enter the liver, namely the hepatic portal vein with dissolved food substances from the small intestine, and the hepatic artery, with oxygenated blood from the lungs. Two ducts originate in the liver, and these unite to form the common hepatic duct which opens, with the pancreatic duct, in the hollow side of the duodenum (the first section of the small intestine). The gall bladder lies inside the liver, and is the storage place for bile, which is formed by the liver cells.

The right lobe of the liver is larger than the left lobe. Each lobe is further divided into many small lobules, each being about the size of a pin-head, and consisting of many liver cells, with bile channels and blood channels between them. A system of blood capillaries, bile capillaries and lymph capillaries runs throughout the entire liver.

The liver cells secrete the bile, and this collects in the bile capillaries, which then unite, forming bile ducts. These bile ducts all eventually unite, forming the main hepatic duct, which gives off a branch, the cystic duct, on its way toward the hepatic duct. The cystic duct leads into the gall bladder. Where a cystic duct joins the hepatic duct, the two continue as the general bile duct, which then joins the pancreatic duct, forming a common duct that opens into the duodenum.

The functions of the liver are varied, working closely with nearly every fundamental system and process in the human body, in particular homeostasis and the regulation of blood sugar.

Regulation of blood sugar: The level of blood sugar stays at around 0.1%, and excess coming from the gut is stored as glycogen. The hormone called insulin—excreted by the pancreas—causes the excess glucose to turn into glycogen.

Regulation of lipids: Lipids are extracted from the blood and changed to carbohydrates, etc. as required or sent to fat storage sites if not needed straight away.

Regulation of amino acids: a supply of amino acids in the blood is kept at a normal level. Any spare which has not been absorbed cannot be stored but is converted into the waste products, called urea when at the liver, and is then sent to the kidneys to be removed from the body as urine. The remainder of the amino acid molecule is not wasted; it is changed into a carbohydrate that can be used.

Production of heat: the liver is one of the hardest working regions of the body and produces a lot of waste heat. This is carried round the body in the blood and warms less active regions.

Forms bile: bile consists of bile salts and the excretory bile pigments. It is important to speed up the digestion of lipids.

Forms cholesterol: this fatty substance is used in the cells. Excess amounts in the blood can cause the blood vessels to become blocked, leading to heart attacks, etc.

Removals of hormones, toxins, etc. The liver extracts many harmful materials from the blood and excretes them in the bile or from the kidneys.

Formation of red blood cells in the young embryo while it is developing in the womb.

Making heparin: this is a substance that prevents the blood from clotting as it travels through the blood system.

Removal of hemoglobin molecules: when red blood cells die, the hemoglobin is converted into bile pigments and the iron atoms are saved for future use.

Storage of blood: the liver can swell to hold huge amounts of blood which can be released into the circulation if the body suddenly needs more, e.g. if it is wounded.

Forms plasma proteins: the plasma proteins are used in blood clotting and in keeping the blood plasma constant. The main blood proteins include fibrinogen, prothrombin, albumens and globulins.

Storage of vitamins such as vitamin A and D. Vitamin A is also made in the liver from carotene, the orange-red pigment in plants. Vitamin B12 is also stored in the liver.

Liver cancer, hepatic cancer, is any cancer of a liver cell, such as a hepatocyte. Liver cancer includes hepatic tumors are tumors or growths on or in the liver. These growths can be benign or malignant (cancerous). There are many forms of liver tumors: such as malignant (cancerous). Most liver cancers are metastases from other tumors, frequently of the GI tract (like colon cancer, carcinoid tumors mainly of the appendix, etc.), but also from breast cancer, ovarian cancer, lung cancer, renal cancer, prostate cancer, etc. The most frequent, malignant, primary liver cancer is hepatocellular carcinoma (also named hepatoma, which is a misnomer because adenomas are usually benign). More rare primary forms of liver cancer include cholangiocarcinoma, mixed tumors, tumors of mesenchymal tissue, sarcoma and hepatoblastoma, a rare malignant tumor in children.

Under the microscope, doctors can distinguish several subtypes of HCC. Most often these subtypes do not affect treatment or prognosis. But one of these subtypes, fibrolamellar, is the most important to recognize. Patients with this rare (less than 1%) type are usually younger (below age 35), and the rest of their liver is not diseased. This subtype has a much better prognosis than other forms of HCC. Cholangiocarcinomas account for about 10% to 20% that start in the liver. They are also called intrahepatic (starting within the liver) cholangiocarcinomas. These cancers start in the small bile ducts (tubes that carry bile to the gallbladder) within the liver.

Angiosarcomas and hemangiosarcomas are rare cancers that begin in blood vessels of the liver. People who have been exposed to vinyl chloride or to thorium dioxide (Thorotrast) are more likely to develop these cancers. Other cases are thought to be due to exposure to arsenic or radium, or to an inherited condition known as hemochromatosis. In about half of all cases, however, no likely cause can be identified. These tumors grow rapidly and are usually too widespread to be removed surgically by the time they are found. Chemotherapy and radiation therapy may not help much. Many patients live less than 6 months after the diagnosis.

Hepatoblastoma is a very rare kind of cancer that develops in children, usually younger than 4 years old. The cells of hepatoblastoma are similar to fetal liver cells. About 70% of children with this disease are treated successfully with surgery and chemotherapy, and the survival rate is greater than 90% for early-stage hepatoblastomas.

Androgen ablation therapy in the treatment of HCC leads to inconsistent results. Disclosed are methods of treating and methods of improving the treatment of HCC. The methods include modulating AR activity wherein the activity is independent from the effects of androgen on AR. Disclosed are mice that lack AR in hepatocytes. This allows for very specific delineation of the effects of AR and of androgen on hepatocytes and abnormal heaptocyte growth and differentiation. By injecting hepatocyte carcinogens into these mice data was produced showing that androgen receptor is involved in liver cancers, such as HCC, but that androgen was not involved. Thus, the control and involvement of AR in liver cancers is independent from the effect of androgen on AR. It was also shown that this androgen independent AR activity was involved in oxidative stress and DNA damage sensing/repairing systems. By inhibiting androgen independent AR activity, such as with ASCJ-9 (5-hydroxy-1,7-bis(3,4-dimethoxyphenyl)-1,4,6-heptatrien-3-one) and derivatives and related molecules which are disclosed in U.S. Pat. Nos. 7,355,081, 6,790,979 and United States Patent Application Publications 20080161391, 20080146660, 20050187255, and 20030203933 and AR siRNA, liver cancer indicators were reduced in in vitro and in vivo models.

As disclosed herein, AR expression was elevated in HCC as compared to normal livers. This leads to methods of diagnoses and prognosis related to assaying the amount of AR expression present in liver tissue, such as a subject's, such as human subject's, liver tissue, such as a hepatocyte. In addition to be just elevated, it is disclosed herein that it is the elevated AR by itself, not the amount of Androgen present, that is indicative of liver cancer presence as well as prognosis when liver cancer is already present in a subject.

AR was up-regulated in dysplastic and HCC human livers. Reduced HCC incidence in mice lacking hepatic AR with little change of serum testosterone. Incidence of HCC induced by DEN higher in male mice than female mice, even when both male and female mice lack AR. This indicates that while assaying for AR as an indicator of presence or progression of liver cancer is appropriate for both male and females, the difference between males and females for HCC likely goes beyond just AR.

It was shown herein that loss of hepatic AR decreases HCC incidence, and loss of hepatic AR results in suppression of HCC growth. Furthermore, loss of hepatic AR results in decreased HCC progression, correlates with lower proliferation, and correlates with higher apoptosis rates. It was also shown herein that loss of hepatic AR increases cell death and apoptosis in the liver tumor during HCC progression.

As shown herein, increased AR results in increased HCC cell growth, and human HCC cells transfected with functional AR result in promotion of cell growth.

Likewise, loss of hepatic AR reduces cellular oxidative stress and decreases DNA damage in the liver, and if one reduces AR one reduces carbonylated groups, reduced oxidized amino acid side chain of protein, at least 30% of control.

Loss of hepatic AR promotes the p53-mediated DNA damage sensing and repairing system and p53-mediated cell apoptosis, and loss of AR up regulates p21, p53 activity, and Gadd45.

It is shown herein that one can suppress carcinogenesis by suppression of cellular oxidative stress and DNA damage and increased p53, results in better DNA sensing and repair, and promotes cell apoptosis.

a) Methods of Screening and Assaying

Disclosed are methods of screening a subject for liver cancer comprising: a) obtaining a tissue sample, and b) assaying for the presence of androgen receptor, wherein the presence of androgen receptor indicates an increased risk of or presence of liver cancer. Also disclosed are methods of testing.

Screening means identifying the presence of a property while testing means determining if a particular property exists.

A subject can be an animal, such as a mammal, such as a primate, such as a human, or a non human, such as an orangutang, a gorilla, a chimpanzee, a monkey, or an animal such as an equine, a dog, a cat, a bovine, an ovine a bird; or a reptile.

Obtaining a tissue sample, for example, can occur using any acceptable way which allows for the tissue to be used in the methods disclosed herein. Typically this means that the tissue will be such that for a period of time the nucleic acids and/or the proteins contained within the cell have not been completely degraded. Typically, less degradation is preferred.

A tissue sample can be any subset of an organism. The sample can be, for example, made up of a portion of an organ, such as a liver. The tissue that is collected can be further subdivided into cells or a cell culture.

Assaying means any method for determining the presence or amount of an object or state such as a protein, such as androgen receptor. For example, assaying for androgen receptor can include identifying the amount of androgen receptor mRNA present in a cell or subset of cells, determining the amount of androgen receptor protein in a cell or subset of cells. The amount can either be determined qualitatively or quantitatively, by for example, using hybridization technology or quantitative PCR, respectively. Methods for determining the amount of mRNA or protein are well understood.

Disclosed are methods, wherein the screening is in a cell, wherein the subject is a mouse, wherein the subject is a human, or wherein the subject is male.

A cell can be can be any cell, such as any liver cell, hepatic cell, hepatocyte.

Presence refers to a detectable amount of a particular object or state. For example, the presence of androgen receptor means that androgen receptor is detectable. The detection of the androgen receptor, in certain instances can be quantified. Often, the presence of an object or state is compared to a control object or state. For example, the presence of androgen receptor can be compared between two different samples or each sample can be compared to a reference amount from a reference standard. For example, as disclosed herein, the presence of androgen receptor is increased in liver cancer cells relative to non-liver cancer cells. As disclosed herein, increase or decrease can be quantified in relative amounts, such as 0.0001 fold, 0.001 fold, 0.005 fold, 0.01 fold, 0.05 fold, 0.1 fold, 0.2 fold, 0.3 fold, 0.4 fold, 0.5 fold, 0.6 fold, 0.7 fold, 0.8 fold, 0.9 fold, 2 fold, 3 fold, 4 fold, 5 fold, 5 fold, 6 fold, 7 fold, 8 fold, 9 fold, 10 fold, 100 fold, 1000 fold, and/or 10000 fold.

### (1) Androgen Receptor

Androgen receptor (AR) is a member of steroid hormone receptor (SHR) family and mediates androgen actions that are involved in a wide range of developmental and physiological responses, such as male sexual differentiation, virilization, and male gonadotropin regulation (Quigley, C. A., et al. 1995. Endocr. Rev. 16:271-321, (Brown, T. R., J Androl 16:299-303 (1995)). Besides its physiological roles, AR also contributes to pathological conditions highlighted by its role in prostate carcinogenesis (Quigley, C. A., et al. 1995. Endocr. Rev. 16:271-321, Santen, R. J. 1992. J. Clin. Endocrinol. Metab. 75:685-689). Like other members of SHR family, the AR contains an amino-terminal (N-terminal) transcription activation domain (TAD, amino acids 1-557 SEQ ID NO: 3 are AF1), a DNA-binding domain (DBD, amino acids 557-623), and a carboxyl-terminal ligand-binding domain (LBD, amino acids 624-919). (AF2 aa 872-908) (Mangelsdorf, D. J., et al., Cell 83:835-9 (1995)). Upon ligand binding, the AR dissociates from chaperone proteins including heat shock proteins, homodimerizes, translocates to the nucleus, and turns on the expression of its target genes by binding to the androgen receptor response element (ARE) (Quigley, C. A., et al. 1995. Endocr. Rev. 16:271-321; Chang, C., A. et al., Crit. Rev Eukaryot Gene Expr 5:97-125 (1995)).

b) AR domains

Compared to the quite conserved DBD and LBD, the N-terminus is quite polymorphic in terms of sequence and length between (nuclear receptors) NRs. The N-terminus is more likely to provide unique surfaces to recruit distinct factors that contribute to the specific action of a certain NR. The AR has a large N-terminus (ARN) and there are two distinct regions important for its transactivation function residing within the ARN: residues 141-338, which are required for full ligand-inducible transactivation, and residues 360-494, where the ligand-independent activation function-1 (AF-1) region is located (Heinlein, C. A., et al. 2002. Endocr. Rev. 23:175-200). Coactivators and corepressors have been identified to interact with ARN (Hsiao, P., et al. 1999. J. Biol. Chem. 274:22373-22379, Hsiao, P., et al. 1999. J. Biol. Chem. 274:20229-20234, Knudsen, K. E., et al. 1999. Cancer Res. 59:2297-2301, Lee, D. K., et al. 2000. J. Biol. Chem. 275:9308-9313, Markus, S. M., et al. 2002. Mol. Biol. Cell 13:670-682, Petre, C. E., et al. 2002. J. Biol. Chem. 277:2207-2215). Furthermore, although ARN extends to more than one half of the full length protein, its associated proteins are relatively fewer compared to those associated with AR DBD and AR LBD, presumably due to the existence of the AF-1 region which limits the application of conventional yeast-two hybrid system by using ARN as bait. It's likely there are still more ARN associated proteins remaining to be identified.

AR is classified with glucocorticoid receptor (GR), mineralocorticoid receptor and progesterone receptor (PR) as one group within the nuclear receptor (NR) superfamily, since they share high homology in the DBD and recognize very similar hormone response elements (Forman, B. M. et al. 1990. Mol. Endocrinol. 4:1293-1301, Laudet, V., et al. 1992. EMBO J. 11:1003-1013). However, the physiological responses mediated by these receptors upon cognate ligand activation are quite distinct and hormone specific. Apparently, these cannot be explained by a specific DNA-binding through the DBD. Factors located outside the DBD may play a key role in determining the specific hormone responses.

2. Coregulators Interact with AR and Other Steroid Receptors

Steroid receptors may function through direct or indirect interaction with other regulatory proteins in cells (McKenna, N. J., and B. W. O'Malley, Cell 108:465-74 (2002); McKenna, N. J., and B. W. O'Malley, Endocrinology 143:2461-5 (2002)). A number of transcriptional coregulators, including coactivators and corepressors, have been identified that enhance or suppress the interactions between steroid receptors and the basal transcriptional machinery (Hermanson, O., et al., Trends Endocrinol Metab 13: 55-60 (2002); 31. Jepsen, K., et al., Cell 102:753-63 (2000); McInerney, E. M., et al., Proc Natl Acad Sci USA 93:10069-73 (1996); Xu, L., et al., Curr Opin Genet Dev 9:140-7 (1999)). It has been suggested that regulation by coregulators is an efficient way to achieve cell- and promoter-specific activation (Pearce, D. et al. 1993. Science 259:1161-1164). A large number of coregulators have been identified in recent years (reviewed in Heinlein, C. A., et al. 2002. Endocr. Rev. 23:175-200, McKenna, N. J., et al. 1999. Endocr. Rev. 20:321-344). For example, SRC-1 can serve as a coactivator to many NRs like PR, estrogen receptor (ER), GR, thyroid hormone receptor (TR) and retinoid X receptor (RXR) (Onate, S. A., et al., Science 270:1354-1357 (1995)). Although NCo-R and SMRT were initially identified to mediate active suppression by unliganded TR and retinoid acid receptor (Chen, J. D., et al. 1995. Nature 377:454-457, Horlein, A. J., et al. 1995. Nature 377:397-404), later studies suggest that they also serve as corepressors to PR (Wagner, B. L., et al. 1998. Mol. Cell. Biol. 18: 1369-1378), ER (Lavinsky, R. M., et al. 1998. Proc. Natl. Acad. Sci. USA 95:2920-2925) and AR (Dotzlaw, H., et al. 2002. Mol. Endocrinol. 16:661-673, Liao, G., et al. 2003. J. Biol. Chem. 278:5052-5061). It is assumed coregulators that can preferentially bind and influence an individual NR at a specific subcellular environment may help to determine the specificity of NR mediated responses.

The p160/steroid receptor coactivator (SRC) family is the most clearly defined class of coactivators, including SRC-1, SRC-2/TIF2, and SRC-3/AIB1/pCIP/RAC3 (Glass, C. K., and M. G. Rosenfeld, Genes Dev 14:121-41 (2000); Llopis, J., et al., Proc Natl Acad Sci USA 97:4363-8 (2000); McKenna, N. J., and B. W. O'Malley, Cell 108:465-74 (2002)). Interaction between ligand-activated steroid receptors and the p160 coactivators is mediated by a small ˜t-helical motif containing the LXXLL sequence (where L is leucine and X is any amino acid) (44). Ligand binding leads to realignment of the helix 12 in the LBD domain revealing a hydrophobic groove where the LXXLL motifs bind (Bledsoe, R. K., et al., Cell 110: 93-105 (2002), Darimont, B. D., et al., Genes Dev 12:3343-56 (1998), Feng, W., et al., Science 280:1747-9 (1998), Heery, D. M., et al., Nature 387:733-6 (1997)). In addition to LXXLL motifs, a number of AR coregulators, such as ARA54 and ARA70, interact with AR in an androgen-dependent manner through FXXLF motifs (where F is phenylalanine) (He, B., et al., J Biol Chem 277:10226-35 (2002), Kang, H. Y., et al., J Biol Chem 274:8570-6 (1999), 63. Yeh, S., and C. Chang, Proc Natl Acad Sci USA 93:5517-21 (1996)). Furthermore, the FXXLF motif located in the AR N-terminal region is found to mediate the interaction between the LBD and N-terminus of AR (N/C interaction), which is important for the full AR transactivation capacity (Chang, C., J. D. et al., Mol Cell Biol 19:8226-39 (1999), He, B., et al., J Biol Chem 275:22986-94 (2000), Langley, E., et al., J Biol Chem 270:29983-90 (1995)). Phage display technique confirms the FXXLF motif is a ligand-dependent AR associated peptide moti (Hsu, C. L., et al., J Biol Chem 278:23691-8 (2003)).

Also disclosed are methods, further comprising the step of comparing the assayed presence of androgen receptor in the tissue sample to a control, wherein more androgen receptor in the tissue sample relative to the control indicates an increased risk of liver cancer.

Also disclosed are methods, wherein the subject has liver cancer, and wherein the presence of androgen receptor indicates a decreased prognosis.

A decreased prognosis means a prognosis that is worse than a prognosis of a control or standard.

Also disclosed are methods of screening a subject for liver cancer comprising: a) obtaining a tissue sample, and b) assaying for the presence of androgen receptor mRNA, wherein the presence of androgen receptor indicates an increased risk of or presence of liver cancer.

Also disclosed are methods, wherein the screening is in a cell, wherein the subject is a mouse, wherein the subject is a human, or wherein the subject is male.

Disclosed are methods of assaying a subject comprising, determining the amount of androgen receptor expressed in a liver cell, and correlating the amount of androgen receptor expressed in the liver cell to the presence of liver cancer in the subject.

Correlating means that the two states or objects are linked in effect. For example, if one state increases, the other state will typically increase. This is called direct correlation. Also, if one state increases and a second state decreases this is called indirect correlation.

Also disclosed are methods, further comprising collecting a sample and then determining the amount of androgen receptor.

Also disclosed are methods, wherein the sample is liver tissue, wherein the sample is a hepatocyte, wherein the step of determining comprises determining the amount androgen receptor RNA present in the cell, wherein the amount of RNA is compared to a control, wherein the amount of RNA is compared to a predetermined standard, wherein the step of determining comprises determining the amount of androgen receptor present, wherein the step of determining comprises using an antibody to androgen receptor. The amount of RNA can be determined by any method for nucleic acid quantification.

Also disclosed are methods, wherein the subject is a male or wherein the subject is a female.

Also disclosed are methods, wherein the proliferation of the liver cancer cells is reduced, wherein the liver cancer cells undergo increased apoptosis, wherein the administration reduces the number of carbonylated groups on amino acids in a liver cell, wherein the administration reduces the number of oxidized amino acid side chains, wherein the reduction is less than 90%, 80%, 70%, 60%, 50%, 40%, 30%, 20%, 10%, 5%, 1%, 0.1%, 0.01%, 0.001% of a control, wherein p21, p53, or Gadd45 are up-regulated.

Disclosed are animal models, wherein the animal model has a disrupted androgen receptor gene, the wherein the disruption occurs specifically in liver cells, wherein the animal is a mouse.

a) Methods of Treating Liver Cancer

Disclosed are methods of treating liver cancer comprising administering to a subject an androgen receptor inhibitor.

An androgen receptor inhibitor means any composition which inhibits the function of androgen receptor, either androgen dependent or androgen independent activity. Androgen dependent or androgen independent activities are as described herein. The androgen receptor inhibitor can be, for example, a functional nucleic acid, as described herein, an antibody, as described herein, or a small molecule inhibitor, such as those described herein. In certain embodiments the androgen receptor is an androgen receptor independent inhibitor which refers to an inhibitor that does not function as an androgen ablation therapy. For example, DHT is not an androgen receptor independent inhibitor.

Disclosed are methods of treating liver cancer comprising administering to a subject a composition, wherein the composition inhibits androgen receptor, wherein the amount of androgen receptor expressed in a liver cell of the subject is assayed.

Also disclosed are methods, wherein the subject has an elevated amount of androgen receptor expressed in a liver cell, wherein the presence of elevated androgen receptor in the subject indicates that the androgen receptor inhibiting composition should be adminstered, wherein after administration of the composition the amount of androgen receptor in a liver cell of the subject is assayed, wherein an additional administration of an androgen receptor inhibiting composition is performed on the subject because the amount of adrogen receptor in the subject's liver cell is elevated, wherein the inhibitor is an androgen receptor independent inhibiting composition.

An elevated amount means more than a control or standard.

Expressed or expression can refer to making mRNA from DNA or making protein from mRNA.

Also disclosed are methods, further comprising administering an oxidative stress inhibiting composition.

Also disclosed are methods, further comprising administering a DNA damage inhibiting composition, wherein the composition comprises a 5-hydroxy-1,7-bis(3,4-dimethoxyphenyl)-1,4,6-heptatrien-3-one) or a derivative, wherein the composition comprises a functional nucleic acid, wherein the functional nucleic acid is an RNAi, wherein the functional nucleic acid is an siRNA, wherein the composition is an AR siRNA, wherein the composition comprises the sequence set forth in SEQ ID NO:11.

Also disclosed are methods, wherein the androgen receptor inhibitor reduces nuclear translocation of androgen receptor, wherein the androgen receptor inhibitor comprises ARA67, or fragment thereof, wherein the androgen receptor inhibitor phosphorylates androgen receptor, wherein the androgen receptor inhibitor comprises GSK2B or fragment thereof, wherein the androgen receptor inhibitor reduces an interaction between the N-terminus and C terminus of androgen receptor, wherein the androgen receptor inhibitor comprises hRad9 or fragment thereof, wherein the androgen receptor inhibitor is ARA67, GSK2B, or hRad9, or fragment thereof, wherein the androgen receptor inhibitor interacts with androgen receptor mRNA, wherein the androgen receptor inhibitor is a functional nucleic acid, wherein the androgen receptor inhibitor is an siRNA, wherein the siRNA comprises SEQ ID NO:11, wherein the cancer is liver cancer, or wherein the subject is a male.

3. Androgen Receptor Signalling

Androgen exerts its effects via the intracellular AR, a member of the superfamily of nuclear receptors (Chang, C. S., et al. (1988) Science 240 (4850), 324-6, Mangelsdorf, D. J., et al. (1995) Cell 83 (6), 835-9). Upon androgen binding, AR dissociates from the heat-shock proteins and binds to androgen response elements (AREs), resulting in upregulation or downregulation of the transcription of AR target genes. In addition to responding to ligands, the AR is affected by kinase signaling pathways which directly or indirectly alter the biological response to androgens. This phenomenon is mediated by the AR, as antiandrogens have been shown to block kinase-induced transcriptional activation (Sadar, M. D. (1999) J Biol Chem 274 (12), 7777-83). Growth factors, cytokines, and neuropeptides have been implicated in various in vitro and in vivo models of human malignancies, including prostate cancers (Burfeind, P., et al. (1996) Proc Natl Acad Sci USA 93 (14), 7263-8). In the absence of androgens, insulin-like growth factor-1 (IGF-1), keratinocyte growth factor (KGF), and epidermal growth factor (EGF) are able to activate transcription of androgen receptor-regulated genes in prostate cancer cells (Culig, Z., et al. (1995) Eur Urol 27 (Suppl 2), 45-7). MAPK and Akt kinase cascades have been shown to be involved in growth factor-mediated AR activation (Yeh, S., et al. (1999) Proc Natl Acad Sci USA 96 (10), 5458-63, Wen, Y., et al. (2000) Cancer Res 60 (24), 6841-5, Lin, H. K., et al. (2001) Proc Natl Acad Sci USA 98 (13), 7200-5). Some neuropeptides, such as bombesin and neurotensin, can stimulate AR activation and cancer cell growth in the absence of androgen, by activation of tyrosine kinase signaling pathways (Lee, L. F., et al. (2001) Mol Cell Biol 21 (24), 8385-97). Prostate cancer cells may progress from androgen-dependence to a refractory state resulting from activation of AR by various kinases, thus circumventing the normal growth inhibition caused by androgen ablation.

This data indicate that AR plays an essential role in the development of liver cancer. Thus, disclosed are assays for diagnosing liver cancer and determining the prognosis of a liver cancer patient by assaying the levels of AR in the liver cancer or cells of the liver cancer subject. Also disclosed are methods of modulating liver cancer by reducing the amount of AR activity in the liver cancer cell. For example, disclosed herein are siRNAs that effectively reduce the AR activity in liver cancer cells and thus, reduce the tumorgenicity of the liver cancer cells, by for example, reducing the ability of the cells to form colonies in a colony forming assay, or reducing the proliferation of the liver cells.

4. AR Activity in General and in Liver Tissue

AR's function as a steroid hormone receptor (SHR) is well documented. Upon binding of its cognate hormone, Androgen, AR dimerizes and is transported into the nucleus where it is able to act on AR specific genes. AR's role in prostate cancer is also well characterized. Androgen ablation therapy, by chemical or physical castration, remains the treatment of choice, but in prostate cancers treated with androgen ablation therapy, using for example, hydroxyflutamide, which is an anti-androgen, blocking productive androgen binding, and thus, decreasing androgen receptor activity, there is typically a refractory period, where the cells become insensitive to the anti-androgen and proliferate in an androgen independent. While there are multiple mechanisms related to this refraction, including mutations in the AR, enhanced expression of growth factor receptors and associated ligands, and overexpression of some AR cofactors, disclosed herein, there is also an underlying androgen independent activity of AR which is involved in, for example, AR's role in liver cancer. Thus, disclosed are methods of modulating AR activity, independent of modulating androgen or its effects on AR, but rather through targeting the androgen independent activity of AR that is now understood to be at least involved in liver cancer.

5. Methods of Inhibiting AR Activity and Inhibiting Cancers Caused by AR Activity

Disclosed are methods of inhibiting AR activity, such as AR activity that is androgen independent, as discussed herein. Typically the methods of inhibiting AR activity involve administering a composition or compound to a cell or organism or in vitro system, such that the compound inhibit activity of the AR, such as the non-androgen dependent activity of AR. Typically, when administering the composition or compound the composition or compound will interact with AR or AR mRNA or other AR nucleic acid, such that, for example the amount of activity AR is decreased (see for example the disclosed siRNA molecules as well as others), the transport of the AR into the nucleus is prevent (See for example, ARA67), the AR is phosphorylated in a region that prevents activity (See for example, GSK3B), of the AR interacts such that interaction between the C and N domains of AR (See for example, hRad9).

It is understood that disclosed herein, there is an interaction between AR and another protein which is required for full AR activity, in for example, liver cancer, where the interaction of AR and the other protein is androgen independent. The methods of inhibiting AR disclosed herein are based on the prevention of this interaction via any of a number of ways, but since the interaction is not dependent on androgen receptor interaction with androgen, antiandrogens, as they have been understood, such as hydroxyflutamide, would not be considered molecules that prevent this non-androgen AR-protein interaction. However, in treating cancers, clearly contemplated would be combination therapies involving antiandrogens, such as hydroxyflutamide, as well as the disclosed AR inhibitors, such as the disclosed AR siRNA molecules or ARA67 or fragments etc.

The compositions can be administered to any animal, including murine, such as mouse and rat and hamster, rabbits, primates, such as chimpanzee, gorilla, orangatan, monkey, or human, ovine, such as sheep and cows, as well as horses.

Disclosed are methods of inhibiting liver cancers comprising administering the disclosed compositions to a cell or an organism or in an in vitro system.

It is also understood that the compositions or compounds can be administered to any type of cell. Typically the compositions and compounds are administered to cells expressing AR and/or AR coregulators, such as co-activators.

Also disclosed are methods for diagnosing cancers caused by AR, such as liver cancer. Disclosed herein, the knowledge that there is an androgen independent activity of AR that is involved in cancer, such as liver cancer, indicates that assaying for the presence of AR, independent, for example, to assaying for the presence of androgen, can be predictive of whether the patient has liver cancer. The connection that AR itself is predictive of cancers, such as liver cancer is made herein. Furthermore, the connection between why AR itself and how AR itself is diagnostic of cancers is also disclosed herein. Thus, disclosed are assays designed to determine the presence of AR protein and/or AR mRNA, for example. Any method for determining protein presence, such as ELISA or antibody hybridization or various chromatographic assays can be used to assay for the presence of androgen receptor in samples, such as a cell or tissue, or organisms, such as a human or other animal disclosed herein. Furthermore, any method for assaying nucleic acid presence, such as hybridization technology, such as probe or chip technology, as well as methods involving amplification, such as reverse transcription/PCR can be used to assay for the presence of androgen receptor in a sample, such as a cell or tissue sample or for its presence in an organism, such as a human or other animal disclosed herein.

Disclosed herein, the effect of AR protein can go through interaction with other protein (s) to have non-genomic and/or non-androgenic activities. AR signals can utilize multiple pathways, including the classic androgen/AR→AR target genes of genomic actions as well as AR→AR interaction proteins of non-genomic action to exert its roles in the liver cancer progression.

6. Human

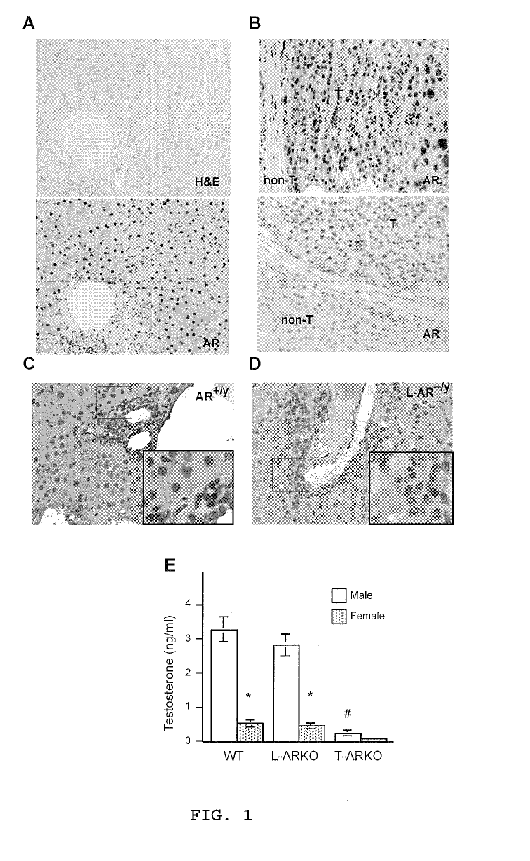

The expression of AR was tested in human livers and higher AR expression was observed in dysplastic liver by using immunohistochemical staining. In addition, higher expression of AR was observed in the HCC lesions that consistent with our findings in the mice experiments. These results are shown in FIGS. 1A, and 1B.

7. Molecules Inhibiting AR Activity

Based on the understanding disclosed herein that AR has activity which is androgen independent, for example, not dependent on the LBD, molecules that target the N-terminal domain as well as the DBD are disclosed herein as inhibitors of AR function, for example, in liver cancer. There are a variety of molecules disclosed herein, having the ability to inhibit AR activity which do not target or depend on the androgen related activity of AR. In other words, the disclosed inhibitors of AR activity will inhibit AR independent of androgen effects. For example, the disclosed inhibitors can be used when, for example, AR has become androgen insensitive and antiandrogens, such as hydroxyflutamide do not work because of the refractory state described herein. Thus, the disclosed inhibitors can be used in combination with antiandrogen therapies. Any means for inhibiting AR can be utilized, because as is disclosed herein, there are activities of AR which are androgen independent and for which inhibition of AR itself, is desirable, not just inhibition of the effects of androgen on AR. For example, molecules disclosed in U.S. Pat. No. 6,790,979 by Lee et al., can be used as described herein, which is herein incorporated by reference in its entirety, but at least for molecules that inhibit AR and their structures.

a) Functional Nucleic Acids

Disclosed are functional nucleic acids that interact with either the mRNA, DNA, or proteins, related to AR, ARA67, GSK2B, and hRad9, for example. In certain embodiments the functional nucleic acids can mimic the binding of, for example, ARA67, GSK2B, or hRad9 to AR, and they will bind AR. In other situations, the functional nucleic acids can mimic the binding of AR to ARA67, GSK2B, or hRad9 binding either ARA67, GSK2B, or hRad9.

b) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of any of the proteins disclosed herein, such as ARA67, GS 2B, or hRad9 or the genomic DNA of any of the proteins disclosed herein, such as ARA67, GSK2B, or hRad9 or they can interact with the polypeptide any of the proteins disclosed herein, such as ARA67, GSK2B, or hRad9. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kds from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of AR, ARA67, GSK2B, hRad9, for example, aptamers, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

c) Protein and Peptides Inhibiting AR

The application discloses a number of proteins and peptides that can inhibit AR. For example, nuclear transport regulators, such as ARA67, can suppress androgen receptor transactivation, phorsphorylation regulators, such as GSK30 and constitutively active forms. Also disclosed are inhibitors of the AR N/C interaction, such as fragments of hRad9.

(1) Antibodies

Disclosed are antibodies that bind the ARA67, AR, GSK2B, or hRad9, for example. In certain embodiments, the antibodies bind AR, such that the antibodies mimic the binding of ARA67, GSK2B, or hRad9 to AR. This mimicking can occur through, for example, competitively binding with ARA 67, GSK2B, or hRad9. These antibodies can be isolated by for example, raising antibodies to AR, as disclosed herein, and then assaying the hybridomas for antibodies that are competed off with ARA67, GSK2B, or hRad9, for example. The antibodies can also be identified by assaying their performance in the disclosed AR activity assays herein, and comparing that activity in the presence of the antibody to, for example, the activity in the presence of ARA67, GSK2B, or hRad9, for example.

(a) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as described herein. The antibodies are tested for their desired activity using the in vitro assays described herein, or by analogous methods, after which their in vivo therapeutic and/or prophylactic activities are tested according to known clinical testing methods.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V (H)) followed by a number of constant domains. Each light chain has a variable domain at one end (V (L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as scFv, sFv, F (ab′)₂, Fab′, Fab and the like, including hybrid fragments. Thus, fragments of the antibodies that retain the ability to bind their specific antigens are provided. For example, fragments of antibodies which maintain ARA67, AR, GSK2B, or hRad9, for example, binding activity or mimic ARA67, AR, GSK2B, or hRad9, for example, binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

(b) Human antibodies

The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147 (1):86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J (H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(c) Humanized antibodies

Optionally, the antibodies are generated in other species and “humanized” for administration in humans. Humanized forms of non-human (e.g., murine) antibodies are chimeric immunoglobulins, immunoglobulin chains or fragments thereof (such as scFv, sFv, Fv, Fab, Fab′, F (ab′)₂, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen (s), is achieved. In general, the CDR residues are directly and most substantially involved in influencing antigen binding (see, WO 94/04679, published 3 Mar. 1994).

(d) Monoclonal Antibodies

The term monoclonal antibody as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain (s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

Monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the complexes described herein.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J (H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147 (1):86-95 (1991)).

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against ARA67, AR, GSK2B, or hRad9, for example. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567 (Cabilly et al.). DNA encoding the monoclonal antibodies of the invention can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

(e) Antibody Fragments

Also disclosed are fragments of antibodies which have bioactivity. The polypeptide fragments of the present invention can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with ARA67, AR, GSK2B, hRad9, TR2, or TR4, for example. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

(f) Administration of Antibodies

Antibodies of the invention are preferably administered to a subject in a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of antibody being administered.

The antibodies can be administered to the subject, patient, or cell by injection (e.g., intravenous, intraperitoneal, subcutaneous, intramuscular), or by other methods such as infusion that ensure its delivery to the bloodstream in an effective form. Local or intravenous injection is preferred.

Effective dosages and schedules for administering the antibodies may be determined empirically, and making such determinations is within the skill in the art. Those skilled in the art will understand that the dosage of antibodies that must be administered will vary depending on, for example, the subject that will receive the antibody, the route of administration, the particular type of antibody used and other drugs being administered. Guidance in selecting appropriate doses for antibodies is found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

(g) Nucleic Acid Approaches for Antibody Delivery

The ARA67, AR, GSK2B, hRad9, TR2, or TR4, for example, antibodies and antibody fragments of the invention can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment.

d) Compositions Identified by Screening with Disclosed Compositions/Combinatorial Chemistry

(1) Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions have the sequences disclosed herein, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, ARA67, AR, GSKB2, or hRad9, for example, are also disclosed. Thus, the products produced using the combinatorial or screening approaches that involve the disclosed compositions, such as, ARA67, AR, GSKB2, or hRad9, for example, are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. No. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94 (23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptidyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94 (23)12997-302 (1997)).

Another preferred method for combinatorial methods designed to isolate peptides is described in Cohen et al. (Cohen B. A., et al., Proc. Natl. Acad. Sci. USA 95 (24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a portion of ARA67, AR, GSKB2, or hRad9, for example, is attached to a DNA binding domain of a transcriptional activation protein, such as Gal 4. By performing the Two-hybrid technique on this type of system, molecules that bind the desired portion of ARA67, AR, GSKB2, or hRad9, for example, can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

Screening molecules similar to ARA67, GSKB2, or hRad9, for example, for inhibition of binding to AR, for example, is a method of isolating desired compounds.

Molecules isolated which bind AR, for example, can either be competitive inhibitors or non-competitive inhibitors of the interaction between AR and ARA67, GSKB2, or hRad9, for example. In certain embodiments the compositions are competitive inhibitors of the interaction between AR and ARA67, GSKB2, or hRad9, for example.

In another embodiment the inhibitors are non-competitive inhibitors of the interaction between AR and ARA67, GSKB2, or hRad9, for example. One type of non-competitive inhibitor will cause allosteric rearrangements which mimic the effect of the interaction between Ar and of the interaction between AR and ARA67, GSKB2, or hRad9, for example.

As used herein combinatorial methods and libraries included traditional screening methods and libraries as well as methods and libraries used in interative processes.

(2) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, AR, ARA67, GSKB2, or hRad9, for example, are also disclosed. Thus, the products produced using the molecular modeling approaches that involve the disclosed compositions, such as, of the interaction between AR, ARA67, GSKB2, or hRad9, for example, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### C. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular AR is disclosed and discussed and a number of modifications that can be made to a number of molecules including the AR are discussed, specifically contemplated is each and every combination and permutation of AR and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Homology/identity

It is understood that one way to define any known variants and derivatives or those that might arise, of the disclosed genes and proteins herein is through defining the variants and derivatives in terms of homology to specific known sequences. For example SEQ ID NO:2 sets forth a particular sequence of an ARA67 and SEQ ID NO:1 sets forth a particular sequence of the protein encoded by SEQ ID NO:2, an ARA67 protein. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

a) Sequences

There are a variety of sequences related to the ARA67, AR, GSK2B, hRad9, TR2, or TR4, for example, and other disclosed genes having the following Genbank Accession Numbers: (SEQ ID NO:1) ARA67 protein, AAH18121; (SEQ ID NO:2) ARA67 DNA, BC018121; (SEQ ID NO:3), AR protein and DNA, NM 000044; (SEQ ID NO:5), GSK3B protein, NP_{—}002084); SEQ ID NO:6 GSK3B DNA, NM-002093); SEQ ID NO:7 hRAD9 protein, AAB39928; SEQ ID NO:8 hRAD 9 cDNA, U53174; SEQ ID NO:13 TR2 protein, M21985; SEQ ID NO:14 TR4 protein, P49116; SEQ ID NO:15 TR2 cDNA, Accession No. M21985; SEQ ID NO:16 TR4 cDNA, P49116, these sequences and others are herein incorporated by reference in their entireties as well as for individual subsequences contained therein.

One particular sequence set forth in SEQ ID NO:3 and having Genbank accession number NM_{—}000044 is used herein, as an example, to exemplify the disclosed compositions and methods. It is understood that the description related to this sequence is applicable to any sequence disclosed herein unless specifically indicated otherwise. Those of skill in the art understand how to resolve sequence discrepancies and differences and to adjust the compositions and methods relating to a particular sequence to other related sequences (i.e. sequences of AR). Primers and/or probes can be designed for any AR sequence given the information disclosed herein and known in the art.

3. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as ARA67, AR, GSK2B, hRad9, TR2, or TR4, for example, into the cell without degradation and include a promoter yielding expression of the gene in the cells into which it is delivered. In some embodiments the ARA67, AR, GSK2B, hRad9, TR2, or TR4, for example, are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virions are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed ARA67, AR, GSK2B, hRad9, TR2, or TR4, for example, or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

5. Peptides

a) Protein Variants

As discussed herein there are numerous variants of the ARA67, AR, GSK2B, hRad9, TR2, or TR4, for example, proteins that are known and herein contemplated. In addition, to the known functional ARA67, AR, GSK2B, hRad9, TR2, or TR4, for example, strain variants there are derivatives of the ARA67, AR, GSK2B, hRad9, TR2, or TR4, for example, proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | Ala A |
| allosoleucine | AIle |
| arginine | Arg R |
| asparagine | Asn N |
| aspartic acid | Asp D |
| cysteine | Cys C |
| glutamic acid | Glu E |
| glutamine | Gln Q |
| glycine | Gly G |
| histidine | His H |
| isolelucine | Ile I |
| leucine | Leu L |
| lysine | Lys K |
| phenylalanine | Phe F |
| proline | Pro P |
| pyroglutamic | pGlu |
| acidp | |
| serine | Ser S |
| threonine | Thr T |
| tyrosine | Tyr Y |
| tryptophan | Trp W |
| valine | Val V |

TABLE 2
Amino Acid Substitutions
Original Residue Exemplary Conservative Substitutions,
others are known in the art.

Ala; Ser
Arg; Lys; Gln
Asn; Gln; His
Asp; Glu
Cys; Ser
Gln; Asn, Lys
Glu, Asp
Gly; Pro
His; Asn; Gln
Ile; Leu; Val
Leu; Ile; Val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

It is understood that one way to define the variants and derivatives of the disclosed proteins herein is through defining the variants and derivatives in terms of homology/identity to specific known sequences. For example, SEQ ID NOs:1, 3, 5, 7, 13, and 14 set forth a particular sequence of ARA67, AR, GSK2B, hRad9, TR2, or TR4 proteins, respectively. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences, such as ARA67, AR, GSK2B, hRad9, TR2, or TR4, for example, it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

6. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution or suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

7. Compositions with Similar Functions

It is understood that the compositions disclosed herein have certain functions, such as binding AR or inhibiting AR function, such as non-androgen related AR activity. Disclosed herein are certain structural requirements for performing the disclosed functions, and it is understood that there are a variety of structures which can perform the same function which are related to the disclosed structures, and that these structures will ultimately achieve the same result, for example, inhibition of non-androgen related AR activity.

### D. METHODS OF MAKING THE COMPOSITIONS

The compositions disclosed herein and the compositions necessary to perform the disclosed methods can be made using any method known to those of skill in the art for that particular reagent or compound unless otherwise specifically noted.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### E. Methods of Using the Compositions

1. Method of Treating Liver Cancer

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1 Example 1

### Androgen Receptor is Therapeutic Target for the Treatment of Hepatocellular Carcinoma

Using Cre-Lox conditional knockout mice model injected with carcinogen, the AR roles in hepatocarcinogenesis were examined. The possible roles of AR in cellular oxidative stress and DNA damage sensing/repairing systems were also tested. Using AR degrading compound, ASCJ-9, or AR-siRNA, the therapeutic potentials of targeting AR in hepatocellular carcinoma (HCC) were examined.

AR expression was elevated in human HCC compared to normal livers. It was found that mice lacking hepatic AR developed later and less HCC than their wild type littermates with comparable serum testosterone in both male and female mice. Addition of functional AR in human HCC cells also resulted in the promotion of cell growth in the absence or presence of 5α-dihydrotestosterone. Mechanistic dissection suggests that AR may promote hepatocarcinogenesis via increased cellular oxidative stress and DNA damage, as well as suppression of p53-mediated DNA damage sensing/repairing system and cell apoptosis. Targeting AR directly via either AR-siRNA or ASC-J9, resulted in suppression of HCC in both ex vivo cell lines and in vivo mice models. The data point to AR, but not androgens, as a therapeutic target for the battle of HCC.

While viral infection and/or environmental carcinogens may lead to the HCC development, the etiology of this liver cancer remains unclear. Early studies suggested that androgens might contribute to the gender difference of HCC incidence and serum testosterone may have a positive linkage to the development of HCC¹. However, clinical trials with targeting of androgens via androgen ablation therapy yield inconsistent and disappointing outcomes².

Androgen effects are mediated mainly through the androgen receptor (AR)³. Androgen/AR signals may modulate many biological events via interaction with various AR coregulators⁴. The biological function of androgen/AR in liver and their detailed consequences before this disclosure, however, remain unclear. The first conditional knockout AR mouse lacking only the hepatic AR (L-AR^{-/y}) was generated via mating floxed-AR mice with albumin promoter-driven Cre-recombinase (Alb-Cre) transgenic mice⁵. Results from these mice in which HCC was induced via injection of N′-N′-diethylnitrosamine (DEN) indicate that the AR, rather than androgens, may play a more dominant role in HCC development.

a) Methods

(1) Human Tissue and IHC Stain

Ten sets of liver tumors (<3 cm) and corresponding normal liver tissues for IHC staining were obtained from ten male patients who received routine liver cancer surgery with inform consent.

(2) Maintenance of Animals and Generation of T-AR−/y, L-AR−/y and T-AR−/−, L-AR−/− mice and inducing HCC using DEN.

All of the animal experiments followed the Guidance of the Care and Use of Laboratory Animals of the NIH with approval from the University of Rochester. The strategy to generate flox-AR gene-targeting mice has been described previously⁶. Briefly, male Actb-Cre or Alb-Cre⁵ (Cre recombinase under control of Albumin promoter; Jackson Lab., B6.Cg-Tg(Alb-cre)21Mgn/J) mice were mated with flox-AR/AR heterozygous female mice to produce T/L-AR^{-/y} male and T/L-AR^{-/+} heterozygous female mice. Another mating using T/L-AR^{-/+} female with AR^{flox/y}/L-AR^{-/y} also generated T/L-AR^{-/-}. 21-day-old pups from tail snips by PCR were genotyped, as described previously⁶. HCC in the liver of 12-day old pups was induced with intraperitoneal (I.P.) injection of a single dose of HCC initiator, DEN (20 mg/kg/mouse; Sigma-Aldrich)¹. After genotyping the pups we divided them into 7 different groups. The groups were 1) AR^{+/y}, 2) T-AR^{-/y}, 3) L-AR^{-/y}, 4) AR^{+/+}, 5) T-AR^{-/-}, 6) L-AR^{-/-}, and 7) AR^{+/y}-untreated with solvent injection only. Several mice from each group were sacrificed at 20-, 24-, 28-, 32-, 36-, and 40 weeks after DEN-injection. The nude mice used for xenograft experiments were 10-weeks-old male nude mice (Charles River; Crl: CD1-Foxn1^{nu} Origin) and ASC-J9 was provided by AndroScience Corporation (San Diego, Calif.).

(3) Serum Testosterone Concentration and Tissue Preservation

Mice at the indicated time points were sacrificed, and 1 ml of blood by cardiocentesis was drawn and immediately assayed for serum testosterone level using the Coat-A-Count Total Testosterone radioimmunoassay (Diagnostic Products). Fresh tissues were flash-frozen in liquid nitrogen for preservation at −80° C. for gene expression assay. We subjected the hepatic major lobe to 10% neutralized buffered formalin (Sigma) for histological analysis.

(4) Histology and Immunohistochemistry

The tissues were fixed in 10% buffered formalin (Sigma) and embedded them in paraffin. For general histologic inspection, tissue sections were treated with Hematoxylin and Eosin (H&E), and then used an ABC kit (Vector Laboratories) to visualize AR, p53, and 8-oxoG (8-oxodeoxyguanosine) immunostaining by specific antibodies (AR (for mice): Santa Cruz, C-19; AR (for human): Dako, 441; p53: Calbiochem, Ab-3; 8-oxoG: Santa Cruz, sc-12075). The TUNEL staining assay was performed (Calbiochem) as previously described⁷. 5′-Bromo-2′-deoxyuridine (BrdU, Sigma) was injected for 4 consecutive days into 55-weeks-old DEN-induced mice. Tissue sections were stained with BrdU specific antibody (Zymed) as previously described⁷.

(5) Statistical Analysis

The results were analyzed using Chi-square tests and Fisher's Exact-tests for cancer incidence using Sigmaplot software, used unpaired T-Test for other experiments, used Standard Deviation (SD) as experimental variation, and considered p-values less than 0.05 to be statistically significant.

(6) Other Methods and Materials

(a) Ex Vivo Cell Culture/Maintenance, Cell Growth, Survival, and Apoptosis Assay

The hepatic cells were isolated from 55-weeks-old DEN-treated mice and performed primary hepatic cell culture as described previously(30). The cells were plated onto 100-mm BioCoat mouse collagen I coated dishes (BD Biosciences) at a density of 5×10⁵ viable cells/dish in low glucose Dulbecco's modified Eagle's medium (Invitrogen) containing 1% bovine serum albumin, 0.8 mM oleate, 0.167 g/ml insulin (4 milliunits/ml), 0.02 g/ml dexamethasone, 100 units of penicillin, and 1% of streptomycin/ml. Human HCC cells were obtained from Dr. Y. S. Jou in Acdemia Secienica, Taiwan published previously(31). The cells were maintained in DMEM (Invitrogen) with 10% Fetal Calf Serum (FCS), 1% Glutamine; and 1% penicillin/streptomycin. The cell growth assays were performed according to a previous study(32) using cell counting assay or MTT (3-(4,5-Dimethylthiazol-2-yl)-2,5-diphenyltetrazolium bromide) assay(33). For cell survival, we treated the cells with various concentrations of H₂O₂ for different times(34), then harvested and measured surviving cells by MTT assay. For apoptotic cells detection, propidium iodine (PI, Sigma-Aldrich) staining was used as described earlier(35). After washing with ice-cold PBS, we re-suspended cells and incubate with PI (1 mg/ml) for 5 min on ice for immediate analysis. The single staining of PI was a positive signal. PI-negative cells were defined as viable while PI-positive cells were apoptotic. The cells were analyzed by flowcytometry using dual-laser FACSCalibur flow cytometer (Becton Dickinson). The AR stable transfectants were established based on a previous procedure(32), obtaining the AR cDNA from the pBabe-AR encoding human AR cDNA sequence, while the AR siRNA construct was from a previously published study(36).

(b) Gene Expression, Reportor Gene as Say, and Oxidative Damage Measurement

The mRNA expression of MnSOD (SOD2), Thioreducin-2, and Gadd45 was determined using quantitative RT-PCR (Q-PCR) as previous described(37), and primer sequences are as follows.

| | |
|---|---|
| Gadd45a, | |
| SEQ ID NO: 24 | 5′-TGAGCTGCTGCTACTGGAGA-3′ |
| SEQ ID NO: 25 | 5′-TGTGATGAATGTGGGTTCGT-3′; |
| Gadd45b, | |
| SEQ ID NO :26 | 5′-ATTGACATCGTCCGGGTATC-3′ |
| SEQ ID NO: 27 | 5′-TGACAGTTCGTGACCAGGAG-3′ |
| MnSOD (SOD2): | |
| SEQ ID NO: 28 | 5′-CTCCAGGCAGAAGCACAG-3′ |
| SEQ ID NO: 29 | 5′-GATATGACCACCACCATTGAA-3′ |
| Thioredoxin2 | |
| SEQ ID NO: 30 | 5′-CAGCCTCTGGCACATTTCCT-3′ |
| SEQ ID NO: 31 | 5′-GTTCGGCTTCTGGTTTCCTTT-3′ |

The p53 expression was analyzed using immunoblotting assay(32) to semi-quantitate p53 protein abundance in the hepatic tumor and HCC cells. The reporter gene assays were performed following previously described procedures(32). The promoter constructs were ARE(4)-Luciferase, Gadd45.-Luciferase, and Thymidine kinase driven renilla luciferase (pRL-TK) which served as transfection efficiency control. Protein abundance was measured by Western blotting as previously described(38) using specific antibodies against AR (Pharmingen), p53 (Cell Signaling), Gadd45, . . . , and Bcl-2 (Santa Cruz). The oxidative stress damage was measured by carbonylated amino acid residue in the protein using Oxiblot kit (Chemicon) following the manufacturer's procedure.

b) Results

(1) AR was Up-Regulated in Dysplastic and HCC Human Livers

The expression of AR in livers from HCC patients was shown. As shown in FIG. 1A, AR expression was highly expressed in a dysplastic liver nodule. Among ten HCC patients examined, Stronger AR expression was found in tumor than surrounding non-tumor in seven patients (FIG. 1B; upper panel). Some of the AR was stained in the border of tumor as shown in FIG. 1B (lower panel). Another patient had AR stained in non-tumor part only.

(2) Generation of L-AR^{-/y}, L-AR^{-/-} and T-AR^{-/y}, T-AR^{-/-} Mice with HCC Development

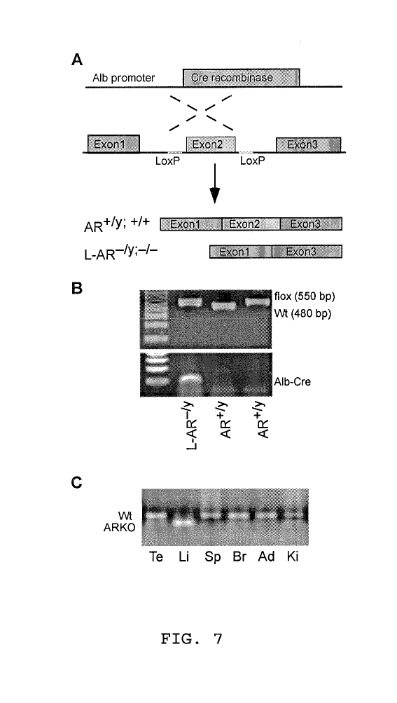

AR knockout mice were generated that are either lacking hepatic AR (L-AR^{-/y}), and their littermates (L-AR^{-/+}) or lacking AR in the whole body (T-AR^{-/y}), and their littermates (T-AR^{-/+}) via mating loxP site-AR female transgene (AR^{flox/flox})⁶ mice with albumin promoter driven (Alb-Cre)⁵ or β-actin promoter driven cre (Actb-Cre)⁶ bearing male transgene mice. (FIG. 7). AR expression was confirmed in nuclei of HCC foci in AR^{+/y} mice, but not in L-AR^{-/y} mice by immunohistochemical staining of AR (FIG. 1C-D).

To develop HCC in these mice, a single injection of the DEN carcinogens was used as described in Methods and separated them into seven groups: 1) AR^{+/y}, 2) L-AR^{-/y}, 3) T-AR^{-/y}, 4) AR^{+/+}, 5) L-AR^{-/-}, 6) T-AR^{-/-}, and 7) untreated male AR^{+/y} mice.

(3) Reduced HCC Incidence in Mice Lacking Hepatic AR with Little Change of Serum Testosterone

The serum testosterone levels remained comparable between 36-weeks DEN-induced L-AR^{-/y} and AR^{+/y}, and between L-AR^{-/-} and AR^{+/+} (normal female mice), even though male mice had much higher serum testosterone levels than female mice. Notably, unlike L-AR^{-/y}, T-AR^{-/y} mice had much lower serum testosterone levels when compared to littermates AR^{+/y} (FIG. 1E).

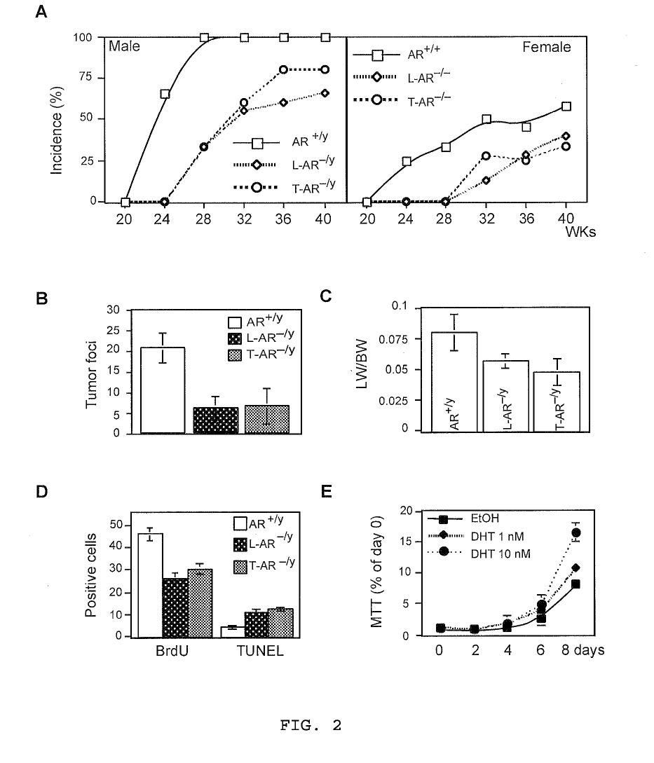

None of the untreated mice (group 7) developed HCC by 40-weeks of age (data not show). In contrast, all other six groups developed HCC with different incidence (FIG. 2A). HCC developed in all the DEN-induced male AR^{+/y} mice examined at 28-, 32-, 36- and 40-weeks of age, whereas only 25-60% of DEN-treated female WT (AR^{+/+}) mice examined at 28˜40-weeks of age developed HCC, confirming the gender-difference in HCC incidence^{1,8}. In contrast, L-AR^{-/y} mice developed less HCC as compared to their WT littermates, even they have comparable serum testosterone. Similar results also occurred in female mice showing L-AR^{-/-} mice developed less HCC with comparable serum testosterone than their WT littermates, suggesting that AR, rather than androgens, is crucial for the development of HCC in both male and female mice. Interestingly, HCC incidence in male L-AR^{-/y} and T-AR^{-/y} mice is still higher than female L-AR^{-/-} and T-AR^{-/-} mice, suggesting factors other than AR might also contribute to the gender-differences in HCC incidence.

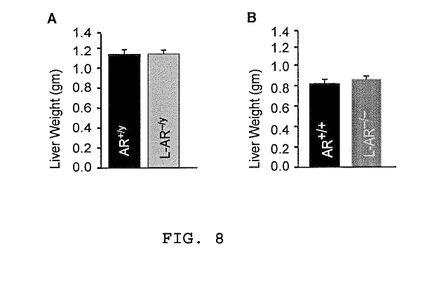

Due to the multiple origin nature of DEN-induced HCC, the numbers of tumor foci were counted and a reduced number of HCC foci in L-AR^{-/y} and T-AR^{-/y} mice were found compared to AR^{+/y} with a ratio of AR^{+/y}: L-AR^{-/y} (or AR^{+/y}: T-AR^{-/y})=20:6 (FIG. 2B). The individual DEN-induced HCC livers were weighed and it was found that the ratio of liver weight to whole body weight (LW/BW) was reduced in L-AR^{-/y} and T-AR^{-/y} mice as compared to their littermate AR^{+/y} mice, indicating that loss of hepatic AR could result in the reduction of HCC tumor mass (FIG. 2C). In contrast, the LW/BW ratio in non-DEN injected L-AR^{-/y} or T-AR^{-/y} mice was similar to their littermate AR^{+/y} mice (FIG. 8), indicating that loss of hepatic AR has little influence on the steady state of normal liver growth in mice without HCC development.

(4) Loss of Hepatic AR Results in Suppression of HCC Growth

Having shown that loss of hepatic AR resulted in reduction of HCC incidence, it was tested whether the loss of hepatic AR might also influence HCC progression that could be correlated with lower proliferation and higher apoptosis rates. Cell proliferation was assessed via intraperitoneal (I.P.) administration of 5′-bromo-2-deoxyuridine (BrdU) in mice for 4 consecutive days. Mice were sacrificed and liver tumors were dissected, embedded, sectioned, and stained with anti-BrdU antibody. Positive stains were counted for proliferate cells and showed the reduction of BrdU (+) staining in both L-AR^{-/y} and T-AR^{-/y} mice as compared to AR^{+/y} mice (FIG. 2D). The TUNEL apoptosis assay was used to measure apoptosis, and it was found that more positive TUNEL staining in L-AR^{-/y} and T-AR^{-/y} as compared to AR^{+/y} mice (FIG. 2D), suggesting that loss of hepatic AR might increase cell death in the liver tumor during HCC progression. Primary cells were isolated from 55-week-old DEN-induced AR^{+/y} mice to examine the androgen 5α-dihydrotestosterone (DHT) effects on cell growth. The results from MTT assay showed that the cell numbers increased in a dose-dependent manner upon DHT treatment (FIG. 2E). Together, using various growth and apoptosis assays, the results (FIG. 2D-E) demonstrated that loss of hepatic AR might lead to the suppression of HCC progression.

(5) Human HCC Cells Transfected with Functional AR Result in Promotion of Cell Growth

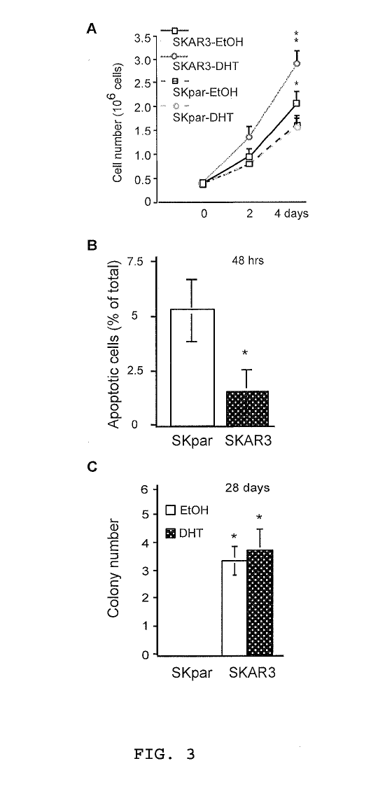

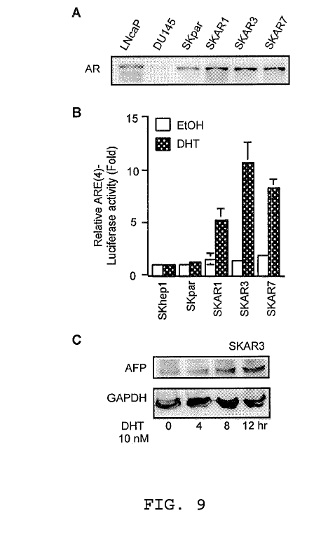

To further strengthen the findings from the mice studies that showed a loss of hepatic AR results in the suppression of HCC growth, human HCC cell lines were used to study the AR effects on HCC cell growth (FIG. 9). Using a cell-counting assay it was shown that DHT had little effect on SKpar (parental transfectant) cell growth (FIG. 3A, SKpar-EtOH vs. SKpar-DHT). In contrast, SKAR3 (stable AR transfectant) increased cell growth (FIG. 3A, SKpar-EtOH vs. SKAR3-EtOH) in the absence of DHT and addition of 10 nM DHT further increased cell growth (FIG. 3A, SKAR3-EtOH vs. SKAR3-DHT). These results indicate that both non-androgen-mediated AR and androgen-mediated AR signals might influence HCC cell growth. Addition of functional AR in SKpar cells also resulted in the decreased cell apoptosis in the absence or presence of DHT (FIG. 3B), suggested that AR, rather than androgen may play more important roles in the hepatic cell apoptosis. This conclusion is further supported with the results from the anchorage-independent cell growth assay. Using soft agar colony formation assay, it was found that SKAR3, but not SKpar cells, were able to grow in an anchorage-independent environment in the absence of androgen, suggesting increased AR expression via transfected functional AR resulted in anchorage-independent cell growth. Addition of 10 nM DHT showed little influence on the AR-promoted anchorage-independent cell growth (FIG. 3C), indicating that the AR, rather than androgen, plays a much more important role for anchorage-independent HCC growth. Together, the results in FIG. 3 indicate that the AR, rather than androgen, plays a more important role in the human HCC cells growth.

(6) Loss of Hepatic AR Reduces Cellular Oxidative Stress and Decreases DNA Damage in the Liver

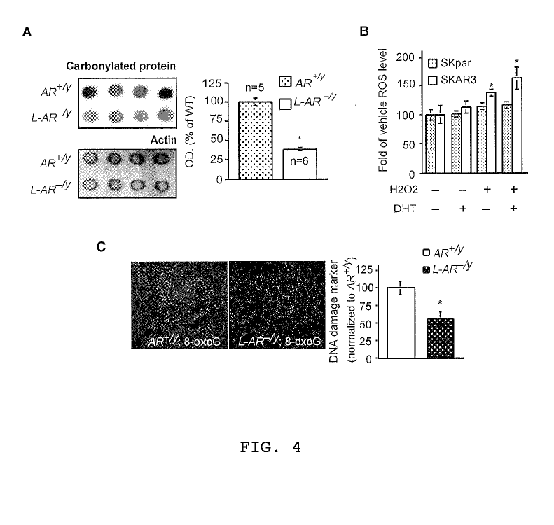

ROS has been linked to the hepatocarcinogenesis during chronic inflammatory liver injury, such as hepatitis and cirrhosis⁹. Early reports also documented the linkage between DEN-induced HCC in mice with innate immune response and the related cellular oxidative stress¹⁰. The cellular oxidative stress levels was evaluated via measuring the carbonylated groups¹¹, the oxidized amino acid side chain of protein (FIG. 4A, upper panels). It was found that cellular ROS levels in the liver tumor of 36-week-old L-AR^{-/y} mice were reduced to 30% as compared to those in DEN-induced AR^{+/y} mice (FIG. 4A, lower panel). To further confirm the effect of androgen/AR signals on cellular ROS level, AR stably-transfected SKAR3 cells were used to examine cellular oxidative stress. The cellular ROS level in SKpar and SKAR3 cells were treated with 250 μM H₂O₂ in the absence or presence of DHT. The results showed that ROS level in SKAR3 cells is increased upon H₂O₂ treatment and further enhanced in the presence of 1 nM DHT as compared to those in SKpar cells (FIG. 4B).

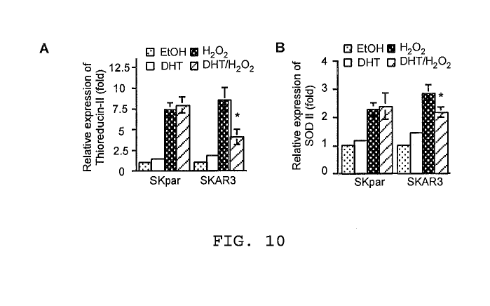

To further dissect how androgen/AR signals may regulate cellular ROS, several key factors that have been linked to ROS were examined and it was found that mRNA expression of thioreducin-2 and superoxide dismutase 2 (SOD2) were decreased after adding 10 nM DHT in SKAR3 cells treated with H₂O₂ (FIG. 10A). In contrast, as there is little functional AR available in SKpar cells, addition of 10 nM DHT failed to suppress the H₂O₂-induced thioreducin-2 and SOD2 mRNA expression (FIG. 10B).

As chronic inflammation induced oxidative stress might result in the breakage or damage of chromosomal DNA, the DNA damage status was examined in mice liver tumors. By staining for the DNA damage marker, 8-oxoG¹², it was found that the positive signal was higher in the liver tumors of AR^{+/y} compared to those in L-AR^{-/y} mice at 36-weeks of DEN induction (FIG. 4C). These results indicate reduced cellular oxidative stress in L-AR^{-/y} mice can suppress the DNA damage, which can then lead to fewer gene mutations and delayed HCC development.

(7) Loss of Hepatic AR Promotes the p53-Mediated DNA Damage Sensing and Repairing System and p53-Mediated Cell apoptosis

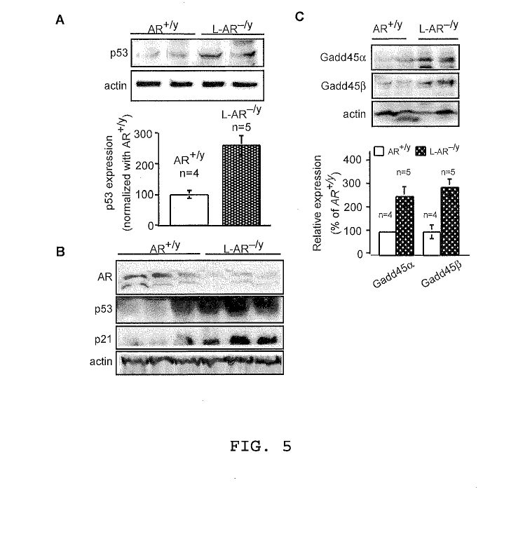

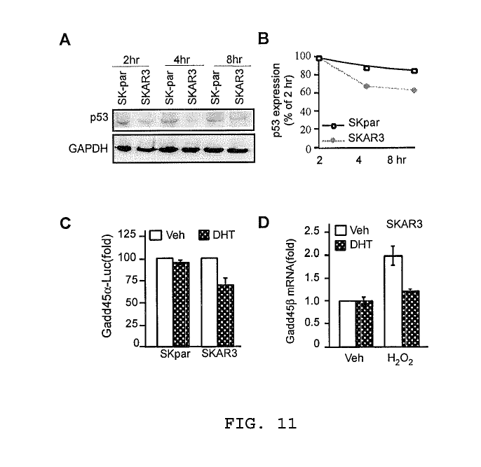

Under normal liver conditions, the increased DNA damage via cellular oxidative stress¹³ can result in the increase of p53-mediated DNA damage sensing and repairing system. The p53 activation can suppress the function of the anti-apoptotic molecule, Bcl-2; therefore triggering an intrinsic cascade for apoptosis¹³. Interestingly, it was found that loss of hepatic AR not only reduced DNA damage, but also enhanced the p53 expression in both normal and liver tumor of L-AR^{-/y} mice (FIG. 5A; 5B). The p53 down stream target gene, p21, was up-regulated in L-AR^{-/y} as well (FIG. 5B). Similar results can be consistently observed in human HCC cells (FIG. 11, 12). Furthermore, enhanced p53 expression might also promote the DNA sensing and repairing system. For example, the expressions of the p53 target gene, Gadd45α¹⁴ and β¹⁵, DNA damage repairing executive genes, were increased in liver tumors of L-AR^{-/y} compared to AR^{-/y} mice (FIG. 5E). It was also found that Gadd45 can be regulated by AR in transcriptional level (FIG. 11). The increased DNA damage sensing and repairing system can then result in the reduced DNA damage seen in liver tumors of L-AR^{-/y} mice. Together, results from FIG. 5 suggested that loss of hepatic AR may suppress hepatocarcinogenesis via 2 pathways: 1) suppression of ROS-induced cellular oxidative stress and DNA damage, and 2) increased p53 expression that results in the better DNA sensing and repairing system as well as promoting cell apoptosis.

(8) Therapeutic Effects on HCC Progression Via Targeting the AR

Based on the above findings showing AR can play a pivotal role for the HCC progression, both ex vivo cells and an in vivo mice model were used to investigate whether AR can be a therapeutic target for the treatment of HCC. Two therapeutic approaches were used: 1) transfection with AR-siRNA, and 2) treatment with the anti-AR compound 5-hydroxy-1,7-bis(3,4-dimethoxyphenyl)-1,4,6-heptatrien-3-one (ASC-J9)

(a) Targeting AR with AR-siRNA

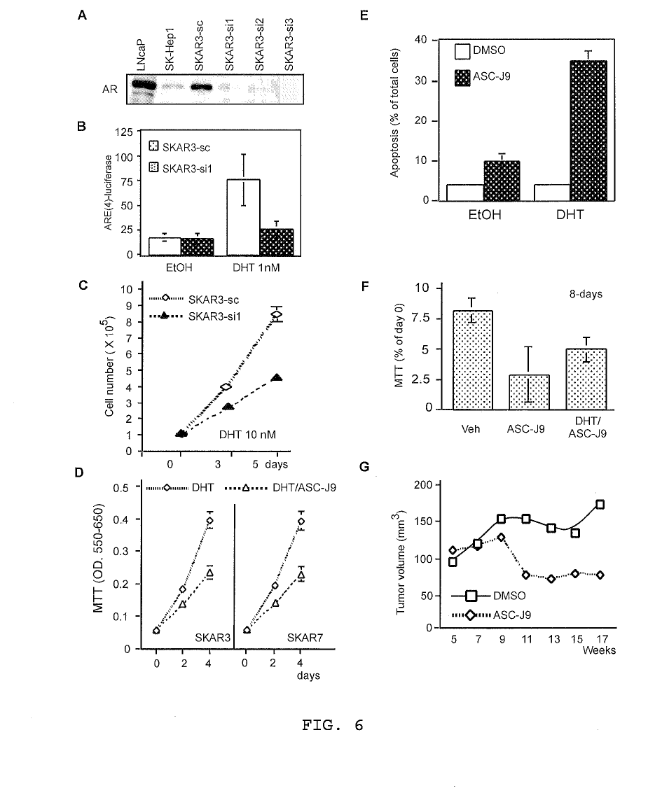

Stable sublines of SKAR3 cells transfected with a retrovirus-based vector that expresses AR-siRNA were established, which effectively knocked down the AR in MCF-7 cells⁷. The AR expression in SKAR3 cells stably-transfected with AR-siRNA (designated SKAR3-si1, 2 or 3) (FIG. 6A) was substantially knocked down. In contrast, AR expressed normally in SKAR3 cells stably transfected with control scramble RNA (designated SKAR3-sc). The effect of the AR-siRNA on the AR-mediated transactivation and AR-mediated cell growth in the stable sublines was investigated. Each stable subline was treated with 1 nM DHT and assessed transactivation by ARE(4)-luciferase promoter assay. It was found that addition of 1 nM DHT could induce substantial AR transactivation in SKAR3-sc, but not SKAR3-si1 cells (FIG. 6B). Using the MTT growth assay, it was also found that knockdown of AR expression via AR-siRNA resulted in the suppression of DHT-induced cell growth (FIG. 6C).

(b) Targeting AR by Treatment with the Anti-AR Compound ASC-J9

The recently developed anti-AR compound ASC-J9 targets AR via dissociating AR from its coregulators, leading to selective degradation of the AR protein. The effects of ASC-J9 on HCC progression in both human HCC cells and in vivo mice model were examined, and it was found that addition of 5 μM ASC-J9 to the SKAR3 and SKAR7 cells resulted in the suppression of cell growth in the presence of 10 nM DHT (FIG. 6D). Furthermore, addition of 5 μM ASC-J9 also resulted in the increased cell apoptosis in the absence or presence of 10 nM DHT (FIG. 6E). This suppression effect on the HCC cell growth was confirmed when human SKAR3 or SKAR7 cells were replaced with primary tumor cells isolated from AR^{+/y} mice livers. It was found that addition of 5 μM ASC-J9 suppressed the primary tumor cell growth in the absence or presence of 10 nM DHT (FIG. 6F). Furthermore, in the mice inoculated with cells isolated from primary liver tumor of AR^{+/y} mice, it was found that I.P. injection of ASC-J9 (50 mg/kg/mice twice per week) resulted in the suppression of tumor growth during the course of 17 weeks treatment (FIG. 6G). Together, results from FIG. 6 suggested that directly targeting the AR either via AR-siRNA or ASC-J9 could suppress HCC progression.

c) Discussion

(1) Up-Regulation of AR Expression in Human HCC Compared to Normal Livers

AR are present in normal liver tissue from both male and female mammalians, but its expression and activation was reported to be increased in the tumor tissue and in the surrounding liver tissue of individuals with HCC¹⁷. Moreover, the expression and activation of AR were reported to be greatly increased in the liver tissue of male and female rodents during chemical-induced liver carcinogenesis¹⁸. In HBV-related HCC, pathways involving androgen-AR signaling, such as serum testosterone concentration, or length of AR CAG length (<23 repeats) may affect the risk of HBV-related HCC among men¹⁹.

(2) AR, but not Androgen is a Better Therapeutic Target for Treatment of HCC

The most important conclusion from these in vivo animal studies with mice lacking hepatic AR and ex vivo studies with human HCC cells transfected with either AR-siRNA or functional AR is a clear demonstration that AR can play pivotal roles for the HCC development and therefore AR, rather than androgens, represents a target for treatment of HCC. The similar findings of AR on hepatocarcinogenesis were also observed in HBV transgene mice with subminimum dosage of DEN injection (unpublished results). This conclusion is against the conventional concept using androgen ablation therapy that only targets androgens is based on the following evidences: 1) Both male and female mice lacking hepatic AR have less HCC incidence with similar serum testosterone compared to the WT littermate mice (FIGS. 1 and 2). 2) Stably transfected functional AR increased cell growth in the absence of DHT (FIG. 3). 3) SKAR3, but not SKpar, cells were able to grow in the absence of androgen in an anchorage-independent environment and addition of 10 nM DHT resulted in little influence of the AR-promoted anchorage-independent cell growth (FIG. 3C). 4) Therapeutic targeting of AR via either AR-siRNA or ASC-J9 resulted in the suppression of HCC progression (FIG. 6) and early data suggested that injection of ASC-J9 for 15 weeks resulted in little change in serum testosterone and mice retained normal sexual function and fertility¹⁶.

### G. REFERENCES

Aberle, H., Bauer, A., Stappert, J., Kispert, A., and Kemler, R. (1997) Embo J 16 (13), 3797-804.
al-Khodairy, F., E. Fotou, K. S. Sheldrick, D. J. Griffiths, A. R. Lehmann, and A. M. Cart. 1994. Identification and characterization of new elements involved in checkpoint and feedback controls in fission yeast. Mol Biol Cell 5:147-60.
Alt, J. R., Cleveland, J. L., Hannink, M., and Diehl, J. A. (2000) Genes Dev 14 (24), 3102-14.
Amundadottir, L. T. and P. Leder. 1998. Signal transduction pathways activated and required for mammary carcinogenesis in response to specific oncogenes. Oncogene 16: 737-46.
Balaji, K. C., Koul, H., Mitra, S., Maramag, C., Reddy, P., Menon, M., Malhotra, R. K., and Laxmanan, S. (1997) Urology 51) (6), 1007-15.
Bayer-Garner, I. B. and B. Smoller. 2000. Androgen receptors: a marker to increase sensitivity for identifying breast cancer in skin metastasis of unknown primary site. Mod Pathol 13:119-22.
Beals, C. R., Sheridan, C. M., Turck, C. W., Gardner, P., and Crabtree, G. R. (1997) Science 275 (5308), 1930-4.
Beitel, L. K., L. Prior, D. M. Vasiliou, B. Gottlieb, M. Kaufman, R. Lumbroso, C. Alvarado, B. McGillivray, M. Trifiro, and L. Pinsky. 1994. Complete androgen insensitivity due to mutations in the probable alpha-helical segments of the DNA-binding domain in the human androgen receptor. Hum Mol Genet. 3:21-7.
Berchem, G. J., M. Bosseler, L. Y. Sugars, H. J. Voeller, S. Zeitlin, and E. P. Gelmann. 1995. Androgens induce resistance to bcl-2-mediated apoptosis in LNCaP prostate cancer cells. Cancer Res 55:735-8.
Berrino, F., P. Muti, A. Micheli, G. Bolelli, V. Krogh, R. Sciajno, P. Pisani, S. Panico, and G. Secreto. 1996. Serum sex hormone levels after menopause and subsequent breast cancer. J Natl Cancer Inst 88: 291-6.
Bessho, T., and A. Sancar. 2000. Human DNA damage checkpoint protein hRAD9 is a 3′ to 5′ exonuclease. J Biol Chem 275:7451-4.
Bieche, I., B. Parfait, S. Tozlu, R. Lidereau, and M. Vidaud. 2001. Quantitation of androgen receptor gene expression in sporadic breast tumors by real-time RT-PCR: evidence that MYC is an AR-regulated gene. Carcinogenesis 22:1521-6.
Birrell, S. N., J. M. Bentel, T. E. Hickey, C. Ricciardelli, M. A. Weger, D. J. Horsfall, and W. D. Tilley. 1995. Androgens induce divergent proliferative responses in human breast cancer cell lines. J Steroid Biochem Mol Biol 52: 459-67.
Bledsoe, R. K., V. G. Montana, T. B. Stanley, C. J. Delves, C. J. Apolito, D. D. McKee, T. G. Consler, D. J. Parks, E. L. Stewart, T. M. Willson, M. H. Lambert, J. T. Moore, K. H. Pearce, and H. E. Xu. 2002. Crystal structure of the glucocorticoid receptor ligand binding domain reveals a novel mode of receptor dimerization and coactivator recognition. Cell 110: 93-105.
Blok, L. J., de Ruiter, P. E., and Brinkmann, A. O. (1998) Biochemistry 37 (11), 3850-7.
Bocchinfuso, W. P., W. P. Hively, J. F. Couse, H. E. Varmus, and K. S. Korach. 1999. A mouse mammary tumor virus-Wnt-1 transgene induces mammary gland hyperplasia and tumorigenesis in mice lacking estrogen receptor-alpha. Cancer Res 59: 1869-76.
Bolla, M., D. Gonzalez, P. Warde, J. B. Dubois, R. O. Mirimanoff, G. Storme, J. Bernier, A. Kuten, C. Sternberg, T. Gil, L. Collette, and M. Pierart. 1997. Improved survival in patients with locally advanced prostate cancer treated with radiotherapy and goserelin. N Engl J Med 337:295-300.
Bonnette, S. G. and D. L. Hadsell. 2001. Targeted disruption of the IGF-I receptor gene decreases cellular proliferation in mammary terminal end buds. Endocrinology 142: 4937-45.
Boyle, W. J., Smeal, T., Defize, L. H., Angel, P., Woodgett, J. R., Karin, M., and Hunter, T. (1991) Cell 64 (3), 573-84.
Brisken, C., A. Ayyannan, C. Nguyen, A. Heineman, F. Reinhardt, J. Tan, S. K. Dey, G. P. Dotto, R. A. Weinberg, and T. January 2002. IGF-2 is a mediator of prolactin-induced morphogenesis in the breast. Dev Cell 3: 877-87.
Brown, T. R. 1995. Human androgen insensitivity syndrome. J Androl 16:299-303.
Brys, M., M. Wojcik, H. Romanowicz-Makowska, and W. M. Krajewska. 2002. Androgen receptor status in female breast cancer: RT-PCR and Western blot studies. J Cancer Res Clin Oncol 128: 85-90.
Burfeind, P., Chemicky, C. L., Rininsland, F., and Ilan, J. (1996) Proc Natl Acad Sci USA 93 (14), 7263-8.
Burn, K., Duggan, B., Atkinson, E. A., Famulski, K. S., Nemer, M., Bleackley, R. C., and Michalak, M. 1994. Modulation of gene expression by calreticulin binding to the glucocorticoid receptor. Nature 367:476-480.
Cai, R. L., Y. Yan-Neale, M. A. Cueto, I-I. Xu, and D. Cohen. 2000. HDAC1, a histone deacetylase, forms a complex with Hus 1 and Rad9, two G2/M checkpoint Rad proteins. J Biol Chem 275:27909-16.
Chang, C. Y., and D. P. McDonnell. 2002. Evaluation of ligand-dependent changes in AR structure using peptide probes. Mol Endocrinol 16:647-60.
Chang, C., A. Saltzman, S. Yeh, W. Young, E. Keller, H. J. Lee, C. Wang, and A. Mizokami. 1995. Androgen receptor: an overview. Crit. Rev Eukaryot Gene Expr 5:97-125.
Chang, C., and Kokontis, J. 1988. Identification of a new member of the steroid receptor super-family by cloning and sequence analysis. Biochem. Biophys. Res. Commun. 155:971-977.
Chang, C., da Silva, S. L., Ideta, R., Lee, Y., Yeh, S., and Burbach, J. P. H. 1994. Human and rat TR4 orphan receptors specify a subclass of the steroid receptor superfamily. Proc. Natl. Acad. Sci. USA 91:6040-6044.
Chang, C., J. D. Norris, H. Gron, L. A. Paige, P. T. Hamilton, D. J. Kenan, D. Fowlkes, and D. P. McDonnell. 1999. Dissection of the LXXLL nuclear receptor-coactivator interaction motif using combinatorial peptide libraries: discovery ofpeptide antagonists of estrogen receptors alpha and beta. Mol Cell Biol 19:8226-39.
Chang, C. S., J. Kokontis, and S. T. Liao. 1988. Molecular cloning of human and rat complementary DNA encoding androgen receptors. Science 240: 324-6.
Chen, J. D., and Evans, R. M. 1995. A transcriptional co-repressor that interacts with nuclear hormone receptors. Nature 377:454-457.
Chen, Y., Martinez, L. A., LaCava, M., Coghlan, L., and Conti, C. J. (1998) Oncogene 16 (15), 1913-20.
Chesire, D. R., Ewing, C. M., Gage, W. R., and Isaacs, W. B. (2002) Oncogene 21 (17), 2679-94.
Coffey, R. N., R. W. Watson, A. J. O'Neill, K. Mc Eleny, and J. M. Fitzpatrick. 2002. Androgen-mediated resistance to apoptosis. Prostate 53:300-9.
Cohen, P., and Frame, S. (2001) Nat Rev Mol Cell Biol 2 (10), 769-76.
Cohen, P., Nimmo, H. G., and Proud, C. G. (1978) Biochem Soe Symp 43, 69-95
Couse, J. F. and K. S. Korach. 1999. Estrogen receptor null mice: what have we learned and where will they lead us? Endocr Rev 211: 358-417.
Cross, D. A., Alessi, D. R., Cohen, P., Andjelkovich, M., and Hemmings, B. A. (1995) Nature 378 (6559), 785-9.
Culbert, A. A., Brown, M. J., Frame, S., Hagen, T., Cross, D. A., Bax, B., and Reith, A. D. (2001) FEBS Lett 507 (3), 288-94.
Culig, Z., Hobisch, A., Cronauer, M. V., Radmayr, C., Trapman, J., Hittmair, A., Bartsch, G., and Klocker, H. (1994) Cancer Res 54 (20), 5474-8.
Culig, Z., Hobisch, A., Cronauer, M. V., Radmayr, C., Trapman, J., Hittmair, A., Bartsch, G., and Klocker, H. (1995) Eur Urol 27 (Suppl 2), 45-7.
Darimont, B. D., R. L. Wagner, J. W. Apriletti, M. R. Stallcup, P. J. Kushner, J. D. Baxter, R. J. Fletterick, and K. R. Yamamoto. 1998. Structure and specificity of nuclear receptor-coactivator interactions. Genes Dev 12:3343-56.
de Groot, R. P., Auwerx, J., Bourouis, M., and Sassone-Corsi, P. (1993) Oncogene 8 (4), 841-7.
Debes, J. D., and D. J. Tindall. 2002. The role of androgens and the androgen receptor in prostate cancer. Cancer Lett 187:1-7.
Dedhar, S., PSR, Shago, M., Hagesteijn, C. L., Yang, H., Filmus, J., Hawley, R. G., Bruchovsky, N., Cheng, H., Matusik, R. J., and Giguere, V. 1994. Inhibition of nuclear hormone receptor activity by calreticulin. Nature 367:480-483.
Di Monaco, M., E. Brignardello, L. Leonardi, V. Gatto, and G. Boccuzzi. 1995. Inhibitory effect of hydroxyflutamide plus tamoxifen on oestradiol-induced growth of MCF-7 breast cancer cells. J Cancer Res Clin Oncol 121:710-4.
Diehl, J. A., Cheng, M., Roussel, M. F., and Sherr, C. J. (1998) Genes Dev 12 (22), 3499-511.
Dimitrakakis, C., J. Zhou, and C. A. Bondy. 2002. Androgens and mammary growth and neoplasia. Fertil Steril 77 Suppl 4: 26-33.
Ding, V. W., Chen, R. H., and McCormick, F. (2000) J Biol Chem 275 (42), 32475-81.
Dotzlaw, H., Moehren, U., Mink, S., Cato, A. C., Iniguez Lluhi, J. A., and Baniahmad, A. 2002. The amino terminus of the human AR is target for corepressor action and antihormone agonism. Mol. Endocrinol. 16:661-673.
Drobnjak, M., Osman, I., Scher, H. I., Fazzari, M., and Cordon-Cardo, C. (2000) Clin Cancer Res 6 (5), 1891-5.
Feldman, B. J., and D. Feldman. 2001. The development of androgen-independent prostate cancer. Nat Rev Cancer 1:34-45.
Feng, W., R. C. Ribeiro, R. L. Wagner, H. Nguyen, J. W. Apriletti, R. J. Fletterick, J. D. Baxter, P. J. Kushner, and B. L. West. 1998. Hormone-dependent coactivator binding to a hydrophobic cleft on nuclear receptors. Science 280:1747-9.
Forman, B. M. and Samuels, H. H. 1990. Interactions among a subfamily of nuclear hormone receptors: the regulatory zipper model. Mol. Endocrinol. 4:1293-1301.
Freire, R., J. R. Mnrguia, M. Tarsounas, N. F. Lowndes, P. B. Moens, and S. P. Jackson. 1998. Human and mouse homologs of Schizosaccharomyces pombe rad 1 (+) and Saccharomyces cerevisiae RAD 17: linkage to checkpoint control and mammalian meiosis. Genes Dev 12:2560-73.
Fu, M., Wang, C., Reutens, A. T., Wang, J., Angeletti, R. H., Siconolfi-Baez, L., Ogryzko, V., Avantaggiati, M., L., and Pestell, R. G. 2000. p300 and p300/cAMP-response element-binding protein-associated factor acetylate the androgen receptor at sites governing hormone-dependent transactivation. J. Biol. Chem. 275:20853-20860.
Fujimoto, N., Yeh, S., Kang, H., Inui, S., Chang, H., Mizokami, A., and Chang, C. 1999. Cloning and characterization of androgen receptor coactivator, ARA55, in human prostate. J. Biol. Chem. 274:8316-8321.
Galigniana, M. D., Hoousley, P. R., DeFranco, D. B., and Pratt, W. B. 1999. Inhibition of glucocorticoid receptor nucleocytoplasmic shuttling by okadaic acid requires intact cytoskeleton. J. Biol. Chem. 275:16222-16227.
Galigniana, M. D., Scruggs, J. L., Herrington, J., Welsh, M. J., Carter-Su, C., Housley, P. R., and Pratt, W. B. 1998. Heat shock protein 90-dependent (geldanamycin-inhibited) movement of the glucocorticoid receptor through the cytoplasm to the nucleus requires intact cytoskeleton. Mol. Endocrinol. 12:1903-1913.
Gao, Y., and Pimplikar, S. W. 2001. The γ-secretase-cleaved C-terminal fragment of amyloid precursor protein mediates signaling to the nucleus. Proc. Natl. Acad. Sci. USA 98:14979-14984.
Gerdes, M. J., Dang, T. D., Larsen, M., and Rowley, D. R. 1998. Transforming growth factor-β1 induces nuclear to cytoplasmic distribution of androgen receptor and inhibits androgen response in prostate smooth muscle cells. Endocrinology 139:3569-3577.
Gittes, R. F. (1991) N Engl J Med 324 (4), 236-45.
Glass, C. K., and M. G. Rosenfeld. 2000. The coregulator exchange in transcriptional functions of nuclear receptors. Genes Dev 14:121-41.
Greene, G. L. 2003. In vivo imaging reveals estrogen receptor's hidden personality. Nat Med 9: 22-3.
Greenlee, R. T., Murray, T., Bolden, S., and Wingo, P. A. (2000) CA Cancer J Clin 50 (1), 7-33.
Greer, D. A., B. D. Besley, K. B. Kennedy, and S. Davey. 2003. hRad9 rapidly binds DNA containing double-strand breaks and is required for damage-dependent topoisomerase II beta binding protein 1 focus formation. Cancer Res 63:4829-35.
Hagmann, M. 1999. Checkpoint gene linked to human cancer. Science 286:2433-4.
Hall, J. L., Chatham, J. C., Eldar-Finkelman, H., and Gibbons, G. H. (2001) Diabetes 50 (5), 1171-9.
Hammond, J., Le, Q., Goodyer, C., Gelfand, M., Trifiro, M., and LeBlanc, A. 2001. Testosteron-mediated neuroprotection through the androgen receptor in human primary neurons. J. Neruochem. 77:1319-1326.
Hang, H., and H. B. Lieberman. 2000. Physical interactions among human checkpoint control proteins HUS 1p, RAD 1p, and RAD9p, and implications for the regulation of cell cycle progression. Genomics 65:24-33.
Hara, T., J. 3/Iiyazaki, It. Araki, M. Yamaoka, N. Kanzaki, M. Kusaka, and M. Miyamoto. 2003. Novel mutations of androgen receptor: a possible mechanism of bicalutamide withdrawal syndrome. Cancer Res 63:149-53.
Hardt, S. E., and Sadoshima, J. (2002) Circ Res 90 (10), 1055-63.
Hartwell, L. H., and M. B. Kastan. 1994. Cell cycle control and cancer. Science 266:1821-8.
Harwood, A. J., Plyte, S. E., Woodgett, J., St-mR, H., and Kay, R. R. (1995) Cell 80 (1), 139-48.
Haslam, S. Z. 1988. Acquisition of estrogen-dependent progesterone receptors by normal mouse mammary gland. Ontogeny of mammary progesterone receptors. J Steroid Biochem 31: 9-13.
He, B., and E. M. Wilson. 2003. Electrostatic modulation in steroid receptor recruitment of LXXLL and FXXLF motifs. Mol Cell Biol 23:2135-50.
He, B., J. A. Kemppainen, and E. M. Wilson. 2000. FXXLF and WXXLF sequences mediate the NH₂-terminal interaction with the ligand binding domain of the androgen receptor. J Biol Chem 275:22986-94.
He, B., J. T. Minges, L. W. Lee, and E. M. Wilson. 2002. The FXXLF motif mediates androgen receptor-specific interactions with coregulators. J Biol Chem 277:10226-35.
He, B., Kemppainen, J. A., and Wilson, E. M. 2000. FXXLF and WXXLF sequences mediate the NH₂-terminal interaction with the ligand binding domain of the androgen receptor. J. Biol. Chem. 275:22986-22994.
He, B., Kemppainen, J. A., Voegel, J. J., Gronemeyer, H., and Wilson, E. M. 1999. Activation function 2 in the human androgen receptor ligand binding domain mediates interdomain communication with the NH₂-terminal domain. J. Biol. Chem. 274:37219-37225.
He, B., Meng, Y. H., and Mivechi, N. F. (1998) Mol Cell Biol 18(11), 6624-33.
Heery, D. M., E. Kalkhoven, S. Hoare, and M. G. Parker. 1997. A signature motif in transcriptional co-activators mediates binding to nuclear receptors. Nature 387:733-6.
Heinlein, C. A., and Chang, C. 2002. Androgen receptor (AR) coregulators: an overview. Endocr. Rev. 23:175-200.
Hermanson, O., C. K. Glass, and M. G. Rosenfeld. 2002. Nuclear receptor coregulators: multiple modes of modification. Trends Endocrinol Metab 13: 55-60.
Hewitt, S. C., W. P. Bocchinfuso, J. Zhai, C. Harrell, L. Koonce, J. Clark, P. Myers, and K. S. Korach. 2002. Lack of ductal development in the absence of functional estrogen receptor alpha delays mammary tumor formation induced by transgenic expression of ErbB2/neu. Cancer Res 62: 2798-805.
Hirai, I., and H. G. Wang. 2002. A role of the C-terminal region of human Rad9 (hRad9) in nuclear transport of the hRad9 checkpoint complex. J Biol Chem 277:25722-7.
Hoeflich, K. P., Luo, J., Rubie, E. A., Tsao, M. S., Jin, O., and Woodgett, J. R. (2000) Nature 406 (6791), 86-90.
Honma, N., G. Sakamoto, F. Akiyama, Y. Esaki, M. Sawabe, T. Arai, T. Hosoi, N. Harada, M. Younes, and K. Takubo. 2003. Breast carcinoma in women over the age of 85: distinct histological pattern and androgen, oestrogen, and progesterone receptor status. Histopathology 42: 120-7.
Horlein, A. J., Naar, A. M., Heinzel, T., Torchia, J., Gloss B., Kurokawa, R., Ryan, A., Kamei, Y., Soderstrom, M., and Glass, C. K. 1995. Ligand-independent repression by the thyroid hormone receptor mediated by a nuclear receptor co-repressor. Nature 377:397-404.
Hsiao, P., and Chang, C. 1999. Isolation and characterization of ARA160 as the first androgen receptor N-terminal-associated coactivator in human prostate cells. J. Biol. Chem. 274:22373-22379.
Hsiao, P., Lin, D., Nakao, R., and Chang, C. 1999. The linkage of Kennedy's neuron disease to ARA24, the first identified androgen receptor polyglutamine region-associated coactivator. J. Biol. Chem. 274:20229-20234.
Hsu, C. L., Y. L. Chen, S. Yeh, H. J. Ting, Y. C. Hu, H. Lin, X. Wang, and C. Chang. 2003. The use of phage display technique for the isolation of androgen receptor interacting peptides with (F/W)XXL (F/W) and FXXLY new signature motifs. J Biol Chem 278:23691-8.
Ikonen, T., Palvimo, J. J., and Janne O. A. 1997. Interaction between the amino and carboxyl terminal regions of rat androgen receptor modulates transcriptional activity and is influenced by nuclear receptor coactivator. J. Biol. Chem. 272:29821-29828.
Inoue, S., A. Orimo, T. Hosoi, S. Kondo, H. Toyoshima, T. Kondo, A. Ikegami, Y. Ouchi, H. Orimo, and M. Muramatsu. 1993. Genomic binding-site cloning reveals an estrogen-responsive gene that encodes a RING finger protein. Proc Natl Acad Sci USA 90: 11117-21.
Jepsen, K., O. Hermanson, T. M. Onami, A. S. Gleiberman, V. Lunyak, R. J. McEvilly, R. Kurokawa, V. Kumar, F. Liu, E. Seto, S. M. Hedriek, G. Mandel, C. K. Glass, D.
W. Rose, and M. G. Rosenfeld. 2000. Combinatorial roles of the nuclear receptor corepressor in transcription and development. Cell 102:753-63.
Jiang, J. G., A. Bell, Y. Liu, and R. Zamegar. 1997. Transcriptional regulation of the hepatocyte growth factor gene by the nuclear receptors chicken ovalbumin upstream promoter transcription factor and estrogen receptor. J Biol Chem 272: 3928-34.
Joon, D. L., M. Hasegawa, C. Sikes, V. S. Khoo, N. H. Terry, G. K. Zagars, M. L. Meistrich, and A. Pollack. 1997. Supraadditive apoptotic response of R3327-G rat prostate tumors to androgen ablation and radiation. Int J Radiat Oncol Biol Phys 38:1071-7.
Kang, H. Y., H. K. Lin, Y. C. Hu, S. Yeh, K. E. Huang, and C. Chang. 2001. From transforming growth factor-beta signaling to androgen action: identification of Smad3 as an androgen receptor coregulator in prostate cancer cells. Proc Natl Acad Sci USA 98:3018-23.
Kang, H. Y., S. Yeh, N. Fujimoto, and C. Chang. 1999. Cloning and characterization of human prostate coactivator ARA54, a novel protein that associates with the androgen receptor. J Biol Chem 274:8570-6.
Kato, S., H. Endoh, Y. Masuhiro, T. Kitamoto, S. Uchiyama, H. Sasaki, S. Masushige, Y. Gotoh, E. Nishida, H. Kawashima, and et al. 1995. Activation of the estrogen receptor through phosphorylation by mitogen-activated protein kinase. Science 270: 1491-4.
Kaur, R., C. F. Kostrub, and T. Enoch. 2001. Structure-function analysis of fission yeast Hus 1-Rad1-Rad9 checkpoint complex. Mol Biol Cell 12:3744-58.
Kleinberg, D. L., M. Feldman, and W. Ruan. 2000. IGF-I: an essential factor in terminal end bud formation and ductal morphogenesis. J Mammary Gland Biol Neoplasia 5: 7-17.
Knudsen, K. E., Cavenee, W. K., and Arden, K. C. 1999. D-type cyclins complex with the androgen receptor and inhibit its transcriptional transactivation ability. Cancer Res. 59:2297-2301.
Kokontis, J., Takakura, K., Hay, N., and Liao, S. (1994) Cancer Res 54 (6), 1566-73.
Komatsu, K., T. Miyashita, H. Hang, K. M. Hopkins, W. Zheng, S. Cuddeback, M. Yamada, H. B. Lieberman, and H. G. Wang. 2000. Human homologue of S. pombe Rad9 interacts with BCL-2/BCL-xL and promotes apoptosis. Nat Cell Biol 2:1-6.
Kostrub, C. F., K. Knudsen, S. Subramani, and T. Enoch. 1998. Huslp, a conserved fission yeast checkpoint protein, interacts with Radlp and is phosphorylated in response to DNA damage. Embo J 17:2055-66.
Krylova, O., Messenger, M. J., and Salinas, P. C. (2000) J Cell Biol 151 (1), 83-94.
Kuenen-Boumeester, V., T. H. Van der Kwast, W. L. van Putten, C. Claassen, B. van Ooijen, and S. C. Henzen-Logmans. 1992. Immunohistochemical determination of androgen receptors in relation to oestrogen and progesterone receptors in female breast cancer. Int J Cancer 52:581-4.
Kuiper, G. G., de Ruiter, P. E., Trapman, J., Boersma, W. J., Grootegoed, J. A., and Brinkmann, A. O. (1993) Biochem J291 (Pt 1), 95-101.
Langley, E., Z. X. Zhou, and E. M. Wilson. 1995. Evidence for an anti-parallel orientation of the ligand-activated human androgen receptor dimer. J Biol Chem 270:29983-90.
Lau, K. F., Miller, C. C., Anderton, B. H., and Shaw, P. C. (1999) J Pept Res 54 (1), 85-91.
Laudet, V., Hanni, C., Coll, J., Catzeflis, F. and Stehelin, D. 1992. Evolution of the nuclear receptor gene superfamily. EMBO J. 11: 1003-1013.
Lavinsky, R. M., Jeppsen, K., Heinzel, T., Torchia, J., Mullen, T., schiff, R., Del-Rio, A., L., Ricote, M., Ngo, S., Gemsch, J., Hilfenbech, S. G., Osborne, C. K., Glass, C. K., Rosenfeld, M. G., and Rose, D. W. 1998. Diverse signaling pathways modulate nuclear receptor recruitment of N-CoR and SMRT complex. Proc. Natl. Acad. Sci. USA 95:2920-2925.
Lea, O. A., S. Kvinnsland, and T. Thorsen. 1989. Improved measurement of androgen receptors in human breast cancer. Cancer Res 49:7162-7.
Lee, D. K., Duan, H. O., and Chang, C. 2000. From androgen receptor to the general transcription factor TFIIH: indication of cdk activating kinase (CAK) as an androgen receptor NH₂-terminal associated coactivator. J. Biol. Chem. 275:9308-9313.
Lee, L. F., Guan, J., Qiu, Y., and Kung, H. J. (2001) Mol Cell Biol 21(24), 8385-97.
Liao, G., Chen, L. Y., Zhang, A., Godavarthy, A., Xia, F., Ghosh, J. C., Li, H., and Chen, J. D. 2003. Regulation of androgen receptor activity by the nuclear receptor corepressor SMRT. J. Biol. Chem. 278:5052-5061.
Lieberman, H. B., K. M. Hopkins, M. Nass, D. Demetrick, and S. Davey. 1996. A human homolog of the Schizosaccharomyces pombe Rad9+ checkpoint control gene. Proc Natl Acad Sci USA 93:13890-5.
Lin, H. K., L. Wang, Y. C. Hu, S. Altuwaijri, and C. Chang. 2002. Phosphorylation-dependent ubiquitylation and degradation of androgen receptor by Akt require Mdm2 E3 ligase. Embo J 21:4037-48.
Lin, H. K., Yeh, S., Kang, H. Y., and Chang, C. (2001) Proc Natl Acad Sci USA 98 (13), 7200-5.
Llopis, J., S. Westin, M. Ricote, Z. Wang, C. Y. Cho, R. Kurokawa, T. M. Mullen, D. W. Rose, M. G. Rosenfeld, R. Y. Tsien, C. K. Glass, and J. Wang. 2000. Ligand-dependent interactions of coactivators steroid receptor coactivator-1 and peroxisome proliferator-activated receptor binding protein with nuclear hormone receptors can be imaged in live cells and are required for transcription. Proc Natl Acad Sci USA 97:4363-8.
Lobaccaro, J. M., S. Lumbroso, C. Belon, F. Galtier-Dereure, J. Bringer, T. Lesimple, M. Namer, B. F. Cutuli, H. Pujol, and C. Sultan. 1993. Androgen receptor gene mutation in male breast cancer. Hum Mol Genet. 2:1799-802.
Lydon, J. P., F. J. DeMayo, C. R. Funk, S. K. Mani, A. R. Hughes, C. A. Montgomery, Jr., G. Shyamala, O. M. Conneely, and B. W. O'Malley. 1995. Mice lacking progesterone receptor exhibit pleiotropic reproductive abnormalities. Genes Dev 9: 2266-78.
Mangelsdorf, D. J., C. Thummel, M. Beato, P. Herrlich, G. Schutz, K. Umesono, B. Blumberg, P. Kastner, M. Mark, P. Chambon, and et al. 1995. The nuclear receptor superfamily: the second decade. Cell 83:835-9.
Markus, S. M., Taneja, S. S., Logan, S. K., Li, W., Ha, S., Hittelman, A. B., Rogatsky, I., and Garabedian, M. J. 2002. Identification and characterization of ART-27, a novel coactivator for the androgen receptor N terminus. Mol. Biol. Cell 13:670-682.
Maucher, A. and E. von Angerer. 1993. Antiproliferative activity of casodex (ICI 176.334) in hormone-dependent tumours. J Cancer Res Clin Oncol 119: 669-74.
McInerney, E. M., D. W. Rose, S. E. Flynn, S. Westin, T. M. Mullen, A. Krones, J. Inostroza, J. Torchia, R. T. Nolte, N. Assa-Munt, M. V. Milburn, C. K. Glass, and M. G. Rosenfeid, 1998. Determinants of coactivator LXXLL motif specificity in nuclear receptor transcriptional activation. Genes Dev 12:3357-68.
McInerney, E. M., M. J. Tsai, B. W. O'Malley, and B. S. Katzenellenbogen. 1996. Analysis of estrogen receptor transcriptional enhancement by a nuclear hormone receptor coactivator. Proc Natl Acad Sci USA 93:10069-73.
McKenna, N. J., and B. W. O'Malley. 2002. Minireview: nuclear receptor coactivators—an update. Endocrinology 143:2461-5.
McKenna, N. J. and B. W. O'Malley. 2002. Combinatorial control of gene expression by nuclear receptors and coregulators. Cell 1118: 465-74.
McKenna, N. J., Lanz, R. B., and O'Malley, B. W. 1999. Nuclear Receptor Coregulators: Cellular and Molecular Biology. Endocr. Rev. 20:321-344.
Meyer, M. E., Gronemeyer, H., Turcotte, B., Bocquel, M. T., Tasset, D., and Chambon, P. 1989. Steroid hormone receptors compete for factors that mediate their enhancer function. Cell 57:433-442.
Migliaccio, A., G. Castoria, M. Di Domenico, A. de Falco, A. Bilancio, M. Lombardi, M. V. Barone, D. Ametrano, M. S. Zannini, C. Abbondanza, and F. Auricchio. 2000. Steroid-induced androgen receptor-oestradiol receptor beta-Src complex triggers prostate cancer cell proliferation. Embo J 19: 5406-17.
Miyoshi, Y., Uemura, H., Fujinami, K., Mikata, K., Harada, M., Kitamura, H., Koizumi, Y., and Kubota, Y. (2000) Prostate 43 (3), 225-32.
Ngan, E. S., Y. Hashimoto, Z. Q. Ma, M. J. Tsai, and S. Y. Tsai. 2003. Overexpression of Cdc25B, an androgen receptor coactivator, in prostate cancer. Oncogene 22:734-9.
Niemann, C., V. Brinkmann, E. Spitzer, G. Hartmann, M. Sachs, H. Naundorf, and W. Birchmeier. 1998. Reconstitution of mammary gland development in vitro: requirement of c-met and c-erbB2 signaling for branching and alveolar morphogenesis. J Cell Biol 143: 533-45.
Nyberg, K. A., R. J. Michelson, C. W. Putnam, and T. A. Weinert. 2002. Toward maintaining the genome: DNA damage and replication checkpoints. Annu Rev Genet. 36:617-56.
Onate, S. A., Tsai, S. Y., Tsai, M. J., and O'Malley, B. W. 1995. Sequence and characterization of a coactivator for the steroid hormone receptor superfamily. Science 270:1354-1357.
Ozanne, D. M., Brady, M. E., Cook, s., Gaughan, L., neal, D. E., and Robson, C. N. 2000. Androgen receptor nuclear translocation is facilitated by the f-actin cross-linking protein filamin. Mol. Endocrinol. 14:1618-1626.
Pan H J, Uno H., Inui S., Fulmer N. O., and Chang C. 1999. Roles of testosterone in the growth of keratinocytes through bald frontal dermal papilla cells. Endocrine 11:321-327.
Pap, M., and Cooper, G. M. (1998) J Biol Chem 273 (32), 19929-32.
Pearce, D. and Yamamoto, K. R. 1993. Mineralocorticoid and glucocorticoid receptor activities distinguished by nonreceptor factors at a composite response element. Science 259:1161-1164.
Perrot-Applanat, M., Lescop, P., and Milgrom, E. 1992. The cytoskeleton and the cellular traffic of the progesterone receptor. J. Cell Biol. 119:337-348.
Petre, C. E., Wetherill, Y. B., Danielsen, M., and Knudsen, K. E. 2002. Cyclin D1: mechanism and consequence of androgen receptor co-repressor activity. J. Biol. Chem. 277:2207-2215.
Pfahl, M. (1993) Endocr Rev 14 (5), 651-8.
Quigley, C. A., A. De Bellis, K. B. Marschke, M. K. el-Awady, E. M. Wilson, and F. S. French. 1995. Androgen receptor defects: historical, clinical, and molecular perspectives. Endocr Rev 16:271-321.
Rahman, M. M., H. Miyamoto, H. Lardy, and C. Chang. 2003. Inactivation of androgen receptor coregulator ARA55 inhibits androgen receptor activity and agonist effect of antiandrogens in prostate cancer cells. Proc Natl Acad Sci USA 100:5124-9.
Rauen, M., M. A. Burtelow, V. M. Dufault, and L. M. Karnitz. 2000. The human checkpoint protein hRadl 7 interacts with the PCNA-like proteins hRadl, hHusl, and hRadg. J Biol Chem 275:29767-71.
Rogatsky, I., Waase, C. L., and Garabedian, M. J. (1998) J Biol Chem 273 (23), 14315-21.
Roos-Mattjus, P., K. M. Hopkins, A. J. Oestreich, B. T. Vroman, K. L. Johnson, S, Naylor, H. B. Lieberman, and L. M. Karnitz. 2003. Phosphorylation of human Rad9 is required for genotoxin-activated checkpoint signaling. J Biol Chem 278:24428-37.
Sadar, M. D. (1999) J Biol Chem 274 (12), 7777-83.
Said, T. K., O. M. Conneely, D. Medina, B. W. O'Malley, and J. P. Lydon. 1997. Progesterone, in addition to estrogen, induces cyclin D1 expression in the murine mammary epithelial cell, in vivo. Endocrinology 138: 3933-9.
Santen, R. J. 1992. Clinical review 37: Endocrine treatment of prostate cancer. J. Clin. Endocrinol. Metab. 75:685-689.
Sears, R., Nuckolls, F., Haura, E., Taya, Y., Tamai, K., and Nevins, J. R. (2000) Genes Dev 14 (19), 2501-14.
Secreto, G. and B. Zumoff. 1994. Abnormal production of androgens in women with breast cancer. Anticancer Res 14:2113-7.
Sells, M. A., U. G. Knaus, S. Bagrodia, D. M. Ambrose, G. M. Bokoch, and J. Chemoff. 1997. Human p21-activated kinase (Pakl) regulates actin organization in mammalian cells. Curr Biol 7: 202-10.
Shaw, M., Cohen, P., and Alessi, D. R. (1997) FEBS Lett 416 (3), 307-11.
Siede, W., A. S. Friedberg, and E. C. Friedberg. 1993. RAD9-dependent G1 arrest defines a second checkpoint for damaged DNA in the cell cycle of Saccharomyces cerevisiae. Proc Natl Acad Sci USA 90:7985-9.
Silberstein, G. B. 2001. Postnatal mammary gland morphogenesis. Microsc Res Tech 52: 155-62.
Simental J. A., Sar M., Lane M. V., French F. S., and Wilson E. M. 1991. Transcriptional activation and nuclear targeting signals of the human androgen receptor. J. Biol. Chem. 266:510-518.
St. Onge, R. P., B. D. Besley, J. L. Pelley, and S. Davey. 2003. A role for the phosphorylation of hRad9 in checkpoint signaling. J Biol Chem 278:26620-8.
Sugimoto, T., S. Stewart, M. Han, and K. L. Guan. 1998. The kinase suppressor of Ras (KSR) modulates growth factor and Ras signaling by uncoupling Elk-1 phosphorylation from MAP kinase activation. Embo J 17:17 17-27.
Sui, G., C. Soohoo, B. Affar el, F. Gay, Y. Shi, and W. C. Forrester. 2002. A DNA vector-based RNAi technology to suppress gene expression in mammalian cells. Proc Natl Acad Sci USA 99: 5515-20.
Szelei, J., J. Jimenez, A. M. Soto, M. F. Luizzi, and C. Sonnenschein. 1997. Androgen-induced inhibition of proliferation in human breast cancer MCF7 cells transfected with androgen receptor. Endocrinology 138: 1406-12.
Thelen, M. P., C. Venclovas, and K. Fidelis. 1999. A sliding clamp model for the Radl family of cell cycle checkpoint proteins. Cell 96:769-70.
Thin, T. H., L. Wang, E. Kim, L. L. Collins, R. Basavappa, and C. Chang. 2003. Isolation and characterization of androgen receptor mutant, AR (M749L), with hypersensitivity to 17-beta estradiol treatment. J Biol Chem 278:7699-708.
Torres, M. A., Eldar-Finkelman, H., Krebs, E. G., and Moon, R. T. (1999) Mol Cell Biol 19 (2), 1427-37.
Truica, C. I., Byers, S., and Gelmann, E. P. (2000) Cancer Res 60 (17), 4709-13.
Urano, T., T. Saito, T. Tsukui, M. Fujita, T. Hosoi, M. Muramatsu, Y. Ouchi, and S. Inoue. 2002. Efp targets 14-3-3 sigma for proteolysis and promotes breast tumour growth. Nature 417:871-5.
Venciovas, C., and M. P. Thelen. 2000. Structure-based predictions ofradl, Radg, Hus 1 and Rad17 participation in sliding clamp and clamp-loading complexes. Nucleic Acids Res 28:2481-93.
Volkmer, E., and L. M. Kamitz. 1999. Human homologs of Schizosaccharomyces pombe rad 1, hus 1, and rad9 form a DNA damage-responsive protein complex. J Biol Chem 274:567-70.
Wagner, B. L., Norris, J. D., Knotts, T. A., Weigel, N. L., and McDonnell, D. P. 1998. The nuclear corepressors NCoR and SMRT are key regulators of both ligand- and 8-bromo-cyclic AMP-dependent transcriptional activity of the human progesterone receptor. Mol. Cell. Biol. 18: 1369-1378.
Wang, Q., Wang, X., Hernandez, A., Hellmich, M. R., Gatalica, Z., and Evers, B. M. (2002) J Biol Chem 24, 24
Wang, X., Yang, Y., Guo, X., Sampson, E. R., Hsu, C. L., Tsai, M. Y., Yeh, S., Wu, G., Guo, Y., and Chang, C. (2002) J Biol Chem 277 (18), 15426-31.
Watson, P. A., K. Kim, K. S. Chen, and M. N. Gould. 2002. Androgen-dependent mammary carcinogenesis in rats transgenic for the Neu proto-oncogene. Cancer Cell 2: 67-79.
Weinert, T. A., and L. H. Hartwell. 1988. The RAD9 gene controls fne ceil cycle response to DNA damage in Saccharomyces cerevisiae. Science 241:317-22.
Wen, Y., Hu, M. C., Makino, K., Spohn, B., Bartholomeusz, G., Yan, D. H., and Hung, M. C. (2000) Cancer Res 60 (24), 6841-5.
Wilson, C. M. and M. J. McPhaul. 1996. A and B forms of the androgen receptor are expressed in a variety of human tissues. Mol Cell Endocrinol 120:51-7.
Woodgett, J. R. (2001) Sci STKE 2001 (100), RE12.
Xie, B., S. W. Tsao, and Y. C. Wong. 1999. Sex hormone-induced mammary carcinogenesis in female noble rats: the role of androgens. Carcinogenesis 20: 1597-606.
Xu, L., C. K. Glass, and M. G. Rosenfeld. 1999. Coactivator and corepressor complexes in nuclear receptor function. Curr Opin Genet Dev 9:140-7.
Yang, L., H. K. Lin, S. Altuwaijri, S. Xie, L. Wang, and C. Chang. 2003. APPL suppresses androgen receptor transactivation via potentiating Akt activity. J Biol Chem 278:16820-7.
Yeh, S, and Chang, C. 1996. Cloning and characterization of a specific coactivator, ARA70, for the androgen receptor in human prostate cells. Proc. Natl. Acad. Sci. USA 93: 5517-5521.
Yeh, S., Lin, H. K., Kang, H. Y., Thin, T. H., Lin, M. F., and Chang, C. (1999) Proc Natl Acad Sci USA 96 (10), 5458-63.
Yeh, S., M. Y. Tsai, Q. Xu, X. M. Mu, H. Lardy, K. E. Huang, H. Lin, S. D. Yeh, S. Altuwaijri, X. Zhou, L. Xing, B. F. Boyce, M. C. Hung, S. Zhang, L. Gan, and C. Chang. 2002. Generation and characterization of androgen receptor knockout (ARKO) mice: an in vivo model for the study of androgen functions in selective tissues. Proc Natl Acad Sci USA 99:13498-503.
Yoshida, K., K. Komatsu, H. G. Wang, and D. Kufe. 2002. c-Abl tyrosine kinase regulates the human Rad9 checkpoint protein in response to DNA damage. Mol Cell Biol 22:3292-300.
Zheng, P. Eastman, J., Pol, S. V., and Pimplikar, S. W. 1998. PAT1, a microtubule-interacting protein, recognizes the basolateral sorting signal of amyloid precursor protein. Proc. Natl. Acad. Sci. USA 95:14745-14750.
Zhou, Z. X., Lane, M. V., Kemppainen, J. A., French, F. S., and Wilson, E. M. 1995. Specificity of ligand-dependent androgen receptor stabilization: receptor domain interactions influence ligand dissociation and receptor stability. Mol. Endocrinol. 9: 208-218.
Zou, L., D. Cortez, and S. J. Elledge. 2002. Regulation of ATR substrate selection by Rad17-dependent loading of Rad9 complexes onto chromatin. Genes Dev 16:198-208.

### VI. Reference

1. Tejura S, Rodgers G R, Dunion M H, Parsons M A, Underwood J C, Ingleton P M. Sex-steroid receptors in the diethylnitrosamine model of hepatocarcinogenesis: modifications by gonadal ablation and steroid replacement therapy. J Mol Endocrinol 1989; 3:229-37.
2. Randomized trial of leuprorelin and flutamide in male patients with hepatocellular carcinoma treated with tamoxifen. Hepatology 2004; 40:1361-9.
3. Chang C S, Kokontis J, Liao S T. Molecular cloning of human and rat complementary DNA encoding androgen receptors. Science 1988; 240:324-6.
4. Heinlein C A, Chang C. Androgen receptor (AR) coregulators: an overview. Endocr Rev 2002; 23: 175-200.
5. Hung-Yun Lin I-CY, Ruey-Shen Wang, Yei-Tsung Chen, Ning-Chun Liu, Saleh Altuwaijri, Cheng-Lung Hsu, Wen-Lung Ma, Jenny Jokinen, Janet D. Sparks, Shuyuan Yeh, Chawnshang Chang. Increased hepatic steatosis and insulin resistance in mice lacking hepatic androgen receptor HEPATOLOGY 2008.
6. Yeh S, Tsai M Y, Xu Q, Mu X M, Lardy H, Huang K E, Lin H, Yeh S D, Altuwaijri S, Zhou X, Xing L, Boyce B F, Hung M C, Zhang S, Gan L, Chang C. Generation and characterization of androgen receptor knockout (ARKO) mice: an in vivo model for the study of androgen functions in selective tissues. Proc Natl Acad Sci USA 2002; 99:13498-503.
7. Yeh S, Hu Y C, Wang P H, Xie C, Xu Q, Tsai M Y, Dong Z, Wang R S, Lee T H, Chang C. Abnormal mammary gland development and growth retardation in female mice and MCF7 breast cancer cells lacking androgen receptor. J Exp Med 2003; 198:1899-908.
8. Ostrowski J L, Ingleton P M, Underwood J C, Parsons M A. Increased hepatic androgen receptor expression in female rats during diethylnitrosamine liver carcinogenesis. A possible correlation with liver tumor development. Gastroenterology 1988; 94:1193-200.
10. Pikarsky E, Porat R M, Stein I, Abramovitch R, Amit S, Kasem S, Gutkovich-Pyest E, Urieli-Shoval S, Galun E, Ben-Neriah Y. NF-kappaB functions as a tumour promoter in inflammation-associated cancer. Nature 2004; 431:461-6.
11. Maeda S, Kamata H, Luo J L, Leffert H, Karin M. IKKbeta couples hepatocyte death to cytokine-driven compensatory proliferation that promotes chemical hepatocarcinogenesis. Cell 2005; 121:977-90.
11. Lim G P, Chu T, Yang F, Beech W, Frautschy S A, Cole G M. The curry spice curcumin reduces oxidative damage and amyloid pathology in an Alzheimer transgenic mouse. J Neurosci 2001; 21:8370-7.
12. Ichinose T, Nobuyuki S, Takano H, Abe M, Sadakane K, Yanagisawa R, Ochi H, Fujioka K, Lee K G, Shibamoto T. Liver carcinogenesis and formation of 8-hydroxy-deoxyguanosine in C3H/HeN mice by oxidized dietary oils containing carcinogenic dicarbonyl compounds. Food Chem Toxicol 2004; 42:1795-803.
13. Hussain S P, Schwank J, Staib F, Wang X W, Harris C C. TP53 mutations and hepatocellular carcinoma: insights into the etiology and pathogenesis of liver cancer. Oncogene 2007; 26:2166-76.
14. Gramantieri L, Chieco P, Giovannini C, Lacchini M, Trere D, Grazi G L, Venturi A, Bolondi L. GADD45-alpha expression in cirrhosis and hepatocellular carcinoma: relationship with DNA repair and proliferation. Hum Pathol 2005; 36:1154-62.
15. Qiu W, David D, Zhou B, Chu P G, Zhang B, Wu M, Xiao J, Han T, Zhu Z, Wang T, Liu X, Lopez R, Frankel P, Jong A, Yen Y. Down-regulation of growth arrest DNA damage-inducible gene 45beta expression is associated with human hepatocellular carcinoma. Am J Pathol 2003; 162:1961-74.
16. Yang Z, Chang Y J, Yu I C, Yeh S, Wu C C, Miyamoto H, Merry D E, Sobue G, Chen L M, Chang S S, Chang C. ASC-J9 ameliorates spinal and bulbar muscular atrophy phenotype via degradation of androgen receptor. Nat Med 2007; 13:348-353.
17. Nagasue N, Ito A, Yukaya H, Ogawa Y. Androgen receptors in hepatocellular carcinoma and surrounding parenchyma. Gastroenterology 1985; 89:643-7.
18. Eagon P K, Elm M S, Epley M J, Shinozuka H, Rao K N. Sex steroid metabolism and receptor status in hepatic hyperplasia and cancer in rats. Gastroenterology 1996; 110:1199-207.
19. Yeh S H, Chiu C M, Chen C L, Lu S F, Hsu H C, Chen D S, Chen P J. Somatic mutations at the trinucleotide repeats of androgen receptor gene in male hepatocellular carcinoma. Int J Cancer 2007; 120:1610-7.
20. Yeh S, Lin H K, Kang H Y, Thin T H, Lin M F, Chang C. From HER2/Neu signal cascade to androgen receptor and its coactivators: a novel pathway by induction of androgen target genes through MAP kinase in prostate cancer cells. Proc Natl Acad Sci USA 1999; 96:5458-63.
21. Miyamoto H, Yang Z, Chen Y T, Ishiguro H, Uemura H, Kubota Y, Nagashima Y, Chang Y J, Hu Y C, Tsai M Y, Yeh S, Messing E M, Chang C. Promotion of bladder cancer development and progression by androgen receptor signals. J Natl Cancer Inst 2007; 99:558-68.
22. Yeh S, Miyamoto H, Chang C. Hydroxyflutamide may not always be a pure antiandrogen. Lancet 1997; 349:852-3.
23. Miyamoto H, Yeh S, Lardy H, Messing E, Chang C. Delta5-androstenediol is a natural hormone with androgenic activity in human prostate cancer cells. Proc Natl Acad Sci USA 1998; 95:11083-8.
24. Yuan J M, Ross R K, Stanczyk F Z, Govindarajan S, Gao Y T, Henderson B E, Yu M C. A cohort study of serum testosterone and hepatocellular carcinoma in Shanghai, China. Int J Cancer 1995; 63:491-3.
25. Yu L, Kubota H, Imai K, Yamaguchi M, Nagasue N. Heterogeneity in androgen receptor levels and growth response to dihydrotestosterone in sublines derived from human hepatocellular carcinoma line (KYN-1). Liver 1997; 17:35-40.
26. Zheng Y, Chen W L, Ma W L, Chang C, Ou J H. Enhancement of gene transactivation activity of androgen receptor by hepatitis B virus X protein. Virology 2007.
27. Lampropoulou-Karatzas C, Goritsas P, Makri MG. Low serum testosterone: a special feature of hepatocellular carcinoma. Eur J Med 1993; 2:23-7.
28. Forbes A, Wilkinson M L, Iqbal M J, Johnson P J, Williams R. Response to cyproterone acetate treatment in primary hepatocellular carcinoma is related to fall in free 5 alpha-dihydrotestosterone. Eur J Cancer Clin Oncol 1987; 23:1659-64.
29. Jie X, Lang C, Jian Q, Chaoqun L, Dehua Y, Yi S, Yanping J, Luokun X, Qiuping Z, Hui W, Feili G, Boquan J, Youxin J, Jinquan T. Androgen activates PEG10 to promote carcinogenesis in hepatic cancer cells. Oncogene 2007.
30. Swift L L, Farkas M H, Major A S, Valyi-Nagy K, Linton M F, Fazio S. A recycling pathway for resecretion of internalized apolipoprotein E in liver cells. J Biol Chem 2001; 276:22965-70.
31. Huang J S, Chao C C, Su T L, Yeh S H, Chen D S, Chen C T, Chen P J, Jou Y S. Diverse cellular transformation capability of overexpressed genes in human hepatocellular carcinoma. Biochem Biophys Res Commun 2004; 315:950-8.
32. Yeh S, Lin H K, Kang H Y, Thin T H, Lin M F, Chang C. From HER2/Neu signal cascade to androgen receptor and its coactivators: a novel pathway by induction of androgen target genes through MAP kinase in prostate cancer cells. Proc Natl Acad Sci USA 1999; 96:5458-63.
33. Mosmann T. Rapid colorimetric assay for cellular growth and survival: application to proliferation and cytotoxicity assays. J Immunol Methods 1983; 65:55-63.
34. Behl C, Skutella T, Lezoualc'h F, Post A, Widmann M, Newton C J, Holsboer F. Neuroprotection against oxidative stress by estrogens: structure-activity relationship. Mol Pharmacol 1997; 51:535-41.
35. Schmid I, Uittenbogaart C H, Giorgi J V. Sensitive method for measuring apoptosis and cell surface phenotype in human thymocytes by flow cytometry. Cytometry 1994; 15:12-20.
36. Yeh S, Hu Y C, Wang P H, Xie C, Xu Q, Tsai M Y, Dong Z, Wang R S, Lee T H, Chang C. Abnormal mammary gland development and growth retardation in female mice and MCF7 breast cancer cells lacking androgen receptor. J Exp Med 2003; 198:1899-908.
37. Yang Z, Chang Y J, Miyamoto H, Yeh S, Yao J L, di Sant'Agnese P A, Tsai M Y, Chang C. Suppression of androgen receptor transactivation and prostate cancer cell growth by heterogeneous nuclear ribonucleoprotein A1 via interaction with androgen receptor coregulator ARA54. Endocrinology 2007; 148:1340-9.
38. Towbin H, Staehelin T, Gordon J. Electrophoretic transfer of proteins from polyacrylamide gels to nitrocellulose sheets: procedure and some applications. Proc Natl Acad Sci USA 1979; 76:4350-4.

| A. | Sequences |
|---|---|
| | 1. SEQ ID NO: 1 |
| | AAH18121. Amyloid beta prec. 585 aa Amyloid |
| | BETA precursor protein-binding protein 2 |
| | [Homo sapiens (ARA67) |
| | 2. SEQ ID NO: 2 |
| | BC018121. Homo sapiens amyl 1758 bp mRNA Homo |
| | sapiens amyloid beta precursor protein |
| | (cytoplasmic tail) binding protein 2, mRNA |
| | complete cds. |
| | 3. SEQ ID NO: 3 |
| | AR protein sequence (Accession No. NM_000044) |
| | 4. SEQ ID NO: 4 |
| | AR cDNA sequence (Accession No. NM_000044) |
| | 5. SEQ ID NO: 5 |
| | GSK3B Protein (Accession No. NP_002084) |
| | 6. SEQ ID NO: 6 |
| | GSK3B DNA (Accession No. NM_002093) |
| | 7. SEQ ID NO: 7 |
| | hRAD9 protein (Accession No. AAB39928) |
| | 8. SEQ ID NO: 8 |
| | hRAD 9 cDNA (Accession No. U53174) |
| | 9. SEQ ID NO: 9 |
| | part of AR siRNA |
| | 10. SEQ ID NO: 10 |
| | Part of AR siRNA |
| | 11. SEQ ID NO: 11 |
| | AR siRNA |
| | Gggcccctgg atggatagct acctcgaggt agctatccat |
| | ccaggggcc |
| | 12. SEQ ID NO: 12 |
| | AR siRNA with poly T after U6 promoter |
| | 13. SEQ ID NO: 13 |
| | TR2 protein (Accession No. M21985) |
| | 14. SEQ ID NO: 14 |
| | TR4 protein (Accession No. P49116) |
| | 15. SEQ ID NO: 15 |
| | TR2 cDNA (Accession No. M21985) |
| | 16. SEQ ID NO: 16 |
| | TR4 cDNA (Accession No. P49116) |
| | 17. SEQ ID NO: 17 |
| | Specific primers for hRAD9, (forward) |
| | 18. SEQ ID NO: 18 |
| | Specific primers for hRAD9, (Reverse) |
| | 19. SEQ ID NO: 19 |
| | 18s rRNA primers, (forward) |
| | 20. SEQ ID NO: 20 |
| | 18s rRNA primers, (reverse) |
| | 21. SEQ ID NO: 21 |
| | Androgen Receptor mutant R614H (AA substitution of |
| | R to H at position 608 |
| | 22. SEQ ID NO: 22 |
| | Small hRad9 peptide |
| | 23. SEQ ID NO: 23 |
| | Small FXXLL peptide |
| | 24. SEQ ID NO: 24 |
| | Gadd45a, 1 |
| 483. | 5′-TGAGCTGCTGCTACTGGAGA-3′ |
| | 25. SEQ ID NO: 25 |
| | Gadd45a, 2 |
| 484. | 5′-TGTGATGAATGTGGGTTCGT-3′; |
| | 26. SEQ ID NO: 26 |
| | Gadd45b, 1 |
| 485. | 5′-ATTGACATCGTCCGGGTATC-3′ |
| | 27. SEQ ID NO: 27 |
| | Gadd45b, 2 |
| 486. | 5′-TGACAGTTCGTGACCAGGAG-3′ |
| | 28. SEQ ID NO: 28 |
| | MuSOD (SOD2):1 |
| 487. | 5′-CTCCAGGCAGAAGCACAG-3′ |
| | 29. SEQ ID NO: 29 |
| | MuSOD (SOD2):2 |
| 488. | 5′-GATATGACCACCACCATTGAA-3′ |
| | 30. SEQ ID NO: 30 |
| | Thioredoxin2 1 |
| 489. | 5′-CAGCCTCTGGCACATTTCCT-3′ |
| | 31. SEQ ID NO: 31 |
| | Thioredoxin2 2 |
| 490. | 5′-GTTCGGCTTCTGGTTTCCTTT-3′ |

What is claimed is:

1. A method of screening a subject for liver cancer comprising: a) obtaining a tissue sample, and b) assaying for the presence of androgen receptor, wherein the presence of androgen receptor indicates an increased risk of or presence of liver cancer.

2. The method of claim 1, wherein the screening is in a cell.

3. The method of claim 1, wherein the subject is a mouse.

4. The method of claim 1, wherein the subject is a human.

5. The method of claim 1, wherein the subject is male.

6. The method of claim 1, further comprising the step of comparing the assayed presence of androgen receptor in the tissue sample to a control, wherein more androgen receptor in the tissue sample relative to the control indicates an increased risk of liver cancer.

7. The method of claim 1, wherein the subject has liver cancer, and wherein the presence of androgen receptor indicates a decreased prognosis.

8. A method of screening a subject for liver cancer comprising: a) obtaining a tissue sample, and b) assaying for the presence of androgen receptor mRNA, wherein the presence of androgen receptor indicates an increased risk of or presence of liver cancer.

9. The method of claim 8, wherein the screening is in a cell.

10. The method of claim 8, wherein the subject is a mouse.

11. The method of claim 8, wherein the subject is a human.

12. The method of claim 8, wherein the subject is male.

13. A method of treating liver cancer comprising administering to a subject an androgen receptor inhibitor.

14. The method of claim 13, wherein the androgen receptor inhibitor reduces nuclear translocation of androgen receptor.

15. The method of claim 13, wherein the androgen receptor inhibitor phosphorylates androgen receptor.

16. The method of claim 13, wherein the androgen receptor inhibitor reduces an interaction between the N-terminus and C terminus of androgen receptor.

17. The method of claim 13, wherein the androgen receptor inhibitor interacts with androgen receptor mRNA.

18. The method of claim 17, wherein the androgen receptor inhibitor comprises a functional nucleic acid.

19. The method of claim 18, wherein the androgen receptor inhibitor comprises an siRNA.

20. The method of claim 19, wherein the siRNA comprises SEQ ID NO:11.

21. The method of claim 13, wherein the cancer is liver cancer.

22. The method of claim 13, wherein the subject is a male.

23. A method of treating liver cancer comprising administering to a subject a composition, wherein the composition inhibits androgen receptor, wherein the amount of androgen receptor expressed in a liver cell of the subject is assayed.

24. The method of claim 23, wherein the subject has an elevated amount of androgen receptor expressed in a liver cell.

25. The method of claim 24, wherein the presence of elevated androgen receptor in the subject indicates that the androgen receptor inhibiting composition should be adminstered.

26. The method of claim 25, wherein after administration of the composition the amount of androgen receptor in a liver cell of the subject is assayed.

27. The method of claim 26, wherein an additional administration of an androgen receptor inhibiting composition is performed on the subject because the amount of adrogen receptor in the subject's liver cell is elevated.

28. The method of claim 23, wherein the androgen receptor independent inhibiting composition.

29. The method of claim 23, further comprising adminstering an oxidative stree inhibiting composition.

30. The method of claim 23, further comprising adminstering a DNA damage inhibiting composition.

31. The method of claim 23, wherein the composition comprises a 5-hydroxy-1,7-bis(3,4-dimethoxyphenyl)-1,4,6-heptatrien-3-one) or a derivative.

32. The method of claim 23, wherein the composition comprises a functional nucleic acid.

33. The method of claim 32, wherein the functional nucleic acid is an RNAi.

34. The method of claim 33, wherein the functional nucleic acid is an siRNA

35. The method of claim 34, wherein the composition is an AR siRNA,

36. The method of claim 35, wherein the composition comprises the sequence set forth in SEQ ID NO:11.

37. A method of assaying a subject comprising, determining the amount of androgen receptor expressed in a liver cell, and correlating the amount of androgen receptor expressed in the liver cell to the presence of liver cancer in the subject.

38. The method of claim 37, further comprising collecting a sample and then determining the amount of androgen receptor.

39. The method of claim 38, wherein the sample is liver tissue.

40. The method of claim 39, wherein the sample is a hepatocyte.

41. The method of claim 37, wherein the step of determining comprises determining the amount androgen receptor RNA present in the cell.

42. The method of claim 41, wherein the amount of RNA is compared to a control.

43. The method of claim 41, wherein the amount of RNA is compared to a predetermined standard.

44. The method of claim 41, wherein the amount of RNA is determined by hybridzation or a nucleic acid amplification method.

45. The method of claim 37, wherein the step of determining comprises determining the amount of androgen receptor present.

46. The method of claim 37, wherein the step of determining comprises using an antibody to androgen receptor.

47. The method of claim 37, wherein the subject is a male.

48. The method of claim 37, wherein the subject is a female.

49. The method of claim 37, wherein the proliferation of the liver cancer cells is reduced.

50. The method of claim 37, wherein the liver cancer cells undergo increased apoptosis.

51. The method of claim 37, wherein the administration reduces the number of carbonylated groups on amino acids in a liver cell.

52. The method of claim 51, wherein the reduction is less than 90%, 80%, 70%, 60%, 50%, 40%, 30%, 20%, 10%, 5%, 1%, 0.1%, 0.01%, 0.001% of a control.

53. The method of claim 37, wherein the administration reduces the number of oxidized amino acid side chains.

54. The method of claim 53, wherein the reduction is less than 90%, 80%, 70%, 60%, 50%, 40%, 30%, 20%, 10%, 5%, 1%, 0.1%, 0.01%, 0.001% of a control.

55. The method of claim 37, wherein p21, p53, or Gadd45 are up-regulated.

56. An animal model, wherein the animal model has a disrupted androgen receptor gene, the wherein the disruption occurs specifically in liver cells.

57. The animal of claim 56, wherein the animal is a mouse.
